(19) United States
(12) Patent Application Publication
(10) Pub. No.: US 2023/0257753 A1
(43) Pub. Date: Aug. 17, 2023

(54) PRODUCTS AND COMPOSITIONS

(71) Applicant: Sirnaomics, Inc., Germantown, MD (US)

(72) Inventor: Dmitry SAMARSKY, Germantown, MD (US)

(21) Appl. No.: 18/170,971

(22) Filed: Feb. 17, 2023

Related U.S. Application Data
(63) Continuation of application No. PCT/US22/47420, filed on Oct. 21, 2022.
(60) Provisional application No. 63/271,057, filed on Oct. 22, 2021.

(51) Int. Cl. C12N 15/113; A61K 45/06

(52) U.S. Cl. CPC C12N 15/1137; A61K 45/06; C12N 2310/14; C12N 2310/351; C12N 2310/315; C12N 2310/51

(57) ABSTRACT

Nucleic acid products that modulate, interfere with, or inhibit PCSK9 and APOC3 gene expression are provided, together with compositions containing the constructs and methods for their use.

### CROSS REFERENCE TO RELATED PATENT APPLICATIONS

This application is a continuation of international application PCT/US2022/47420, filed Oct. 21, 2022, which claims the benefit of and priority to U.S. Provisional Patent Application No. 63/271,057, filed Oct. 22, 2021, the contents of each of which are incorporated herein by reference in their entireties.

### SEQUENCE LISTING

The instant application contains a sequence listing, submitted electronically in ST.26 (XML) format, is hereby incorporated by reference in its entirety. The ST.26 sequence listing, created on Dec. 8, 2022, is named 4690_0056I_SL and is 2674 kilobytes in size.

### FIELD

Nucleic acid products are provided that modulate, interfere with, and/or inhibit PCSK9 and APOC3 gene expression. Methods, compounds, and compositions are provided for reducing expression of PCSK9 and APOC3 mRNA and protein in an animal. Such methods, compounds, and compositions are useful to treat, prevent, or ameliorate PCSK9- and APOC3-associated disorders such as dyslipidemia, more specifically hypercholesterinemia, hypertriglyceridemia, hyperchylomicronemia, and atherosclerotic cardiovascular disease (ASCVD).

### BACKGROUND

Cholesterol has multiple vital functions, including the maintenance of integrity and fluidity of cell membranes. It furthermore serves a precursor for biosynthetic pathways, including those leading to steroid hormones and vitamin D. Cholesterol is present in the blood, where it occurs mainly in two forms: as component in high density lipoproteins (HDL) and in low density lipoproteins (LDL). Often the HDL cholesterol is referred as “good” or beneficial, while LDL cholesterol, especially when present in elevated levels, presents a health risk and lead to disease. Proprotein convertase subtilisin/kexin type 9 (PCSK9) is a serine protease involved in lipid metabolism. PCSK9 reduces the number of LDL receptors on the surface of liver cells. As a consequence, elevated amounts and/or activity of PCSK9 entail higher blood levels of “bad” LDL cholesterol. This molecular and cellular function of PCSK9 has led to its recognition as a therapeutic target.

Triglycerides are esters of glycerol with three fatty acids. They serve as storage of fat and energy and are transported via the bloodstream. Excess level of blood triglycerides have been recognized early on as causative agents or bystanders of a range of disorders. More recent evidence suggests a causative role, partly in conjunction with elevated levels of cholesterol (especially LDL cholesterol) in ASCVD and disorders subsumed under this term or associated therewith. A more comprehensive list of disorders associated with elevated levels of triglycerides is given in the embodiments disclosed further below.

Apolipoprotein C3 is secreted by the liver and the small intestine. It can be found on triglyceride-rich lipoproteins including very low density lipoproteins (VLDL) and chylomicrons. It is involved in the negative regulation of lipid catabolism, especially triglyceride catabolism, and of the clearance of VLDL, LDL and HDL lipoproteins. A molecular function of APOC3 is the inhibition of lipoprotein lipase and of hepatic lipase.

### Disease

Abnormal amounts of circulating cholesterol, in particular of LDL cholesterol, also referred to as hypercholesterolemia, is a recognized disorder in itself which is inter alia owed to the fact that such abnormal amounts, especially if they persist over extended periods of time, may entail disorder of the cardiovascular system. More specifically, excess amounts of cholesterol may deposit on the inner walls of blood vessel which in turn may lead to clogging, where the clinical manifestations of such clogging include myocardial infarction, stroke and peripheral artery disease.

Also abnormal amounts of circulating triglycerides, also referred to as hypertriglyceridemia, is a recognized disorder in itself which is inter alia owed to the fact that such abnormal amounts, especially if they persist over extended periods of time, may entail disorders of the cardiovascular system and/or inflammation.

### Treatment

Established treatments for the exemplified disorder disclosed above include the administration of statins. However, statins may cause side effects, and certain patients are statin-intolerant. There therefore remains a need for therapies to treat lipid dysregulation and associated diseases including PCSK9- and APOC3-associated diseases. We, therefore, aim to provide compounds, methods, and pharmaceutical compositions for the treatment of such diseases.

Double-stranded RNA (dsRNA) able to complementarily bind expressed mRNA has been shown to be able to block gene expression (Fire et al, 1998, Nature. 1998 Feb. 19; 391 (6669):806-1 1 and Elbashir et at., 2001, Nature. 2001 May 24; 41 1 (6836):494-8) by a mechanism that has been termed RNA interference (RNAi). Short dsRNAs direct gene-specific, post-transcriptional silencing in many organisms, including vertebrates, and have become a useful tool for studying gene function. RNAi is mediated by the RNA-induced silencing complex (RISC), a sequence-specific, multi-component nuclease that destroys messenger RNAs homologous to the silencing trigger loaded into the RISC complex. Interfering RNA (iRNA) such as siRNAs, antisense RNA, and micro-RNA are oligonucleotides that prevent the formation of proteins by gene-silencing i.e., inhibiting gene translation of the protein through degradation of mRNA molecules. Gene-silencing agents are becoming increasingly important for therapeutic applications in medicine.

According to Watts and Corey in the Journal of Pathology (2012; Vol 226, p 365-379) there are algorithms that can be used to design nucleic acid silencing triggers, but all of these have severe limitations. It may take various experimental methods to identify potent siRNAs, as algorithms do not take into account factors such as tertiary structure of the target mRNA or the involvement of RNA binding proteins. Therefore, the discovery of a potent nucleic acid silencing trigger with minimal off-target effects is a complex process. For the pharmaceutical development of these highly charged molecules it is necessary that they can be synthesised economically, distributed to target tissues, enter cells and function within acceptable limits of toxicity. An aim is to, therefore, provide compounds, methods, and pharmaceutical compositions for the treatment of thromboembolic diseases as described herein, which comprise oligomeric compounds that modulate, in particular inhibit, gene expression by RNAi.

### SUMMARY

Nucleic acid products are provided that modulate, interfere with or inhibit, PCSK9 and APOC3 gene expression, and associated therapeutic uses. Specific oligomeric compounds and sequences are described herein.

### BRIEF DESCRIPTION OF THE FIGURES

FIG. 1a shows a concentration dependence of PCSK9 in vitro inhibition by certain muRNA constructs of Table 7a, and by other compounds (TMPRSS6, 91conv31A and MK4K4 G8) as a negative control.

FIG. 1b shows a concentration dependence of APOC3 in vitro inhibition by certain muRNA constructs of Table 7a, and by other compounds (TMPRSS6, 91conv31A and MK4K4 G8) as a negative control.

FIG. 1c shows a concentration dependence of PCSK9 in vitro inhibition by certain muRNA constructs of Table 7b, and by other compound M4K4 G8 as a negative control and PC1aas a reference compound.

FIG. 1d shows a concentration dependence of APOC3 in vitro inhibition by certain muRNA constructs of Table 7b, and by other compound M4K4 G8 as a negative control and PC1aas a reference compound.

FIG. 1e shows a concentration dependence of PCSK9 in vitro inhibition by muRNA construct A28-P44 versus PC1 and TMPRSS6.

FIG. 1f shows a concentration dependence of APOC3 in vitro inhibition by muRNA construct A28-P44 versus PC1 and TMPRSS6.

FIG. 2a shows in vivo inhibition of APOC3 expression in mice using certain muRNA constructs of Table 7b.

FIG. 2b shows in vivo inhibition of PCSK9 expression in mice using certain muRNA constructs of Table 7b.

### DETAILED DESCRIPTION AND EMBODIMENTS

The following aspects are non-limiting:

Aspect 1. A nucleic acid construct comprising at least:

(a) a first nucleic acid portion that is at least partially complementary to at least a first portion of an RNA which is transcribed from a PCSK9 gene;

(b) a second nucleic acid portion that is at least partially complementary to at least a second portion of an RNA which is transcribed from a APOC3 gene;

(c) a third nucleic acid portion that is at least partially complementary to said first nucleic acid portion of (a), so as to form a first nucleic acid duplex region therewith;

(d) a fourth nucleic acid portion that is at least partially complementary to said second nucleic acid portion of (b), so as to form a second nucleic acid duplex region therewith.

Aspect 2. A composition comprising a construct according to aspect 1, and a physiologically acceptable excipient.

Aspect 3. A pharmaceutical composition comprising a construct according to aspect 1.

Aspect 4. A construct according to aspect 1, for use in human or veterinary medicine or therapy.

Aspect 5. A construct according to aspect 1, for use in a method of treating a disease or disorder.

Aspect 6. A method of treating a disease or disorder comprising administration of a construct according to aspect 1, to an individual in need of treatment.

Aspect 6a. A use of a nucleic acid construct according to aspect 1 in the manufacture of a medicament for a treatment of a disease or disorder.

Aspect 7. Use of a construct according to aspect 1, for use in research as a gene function analysis tool.

Further embodiments (items) are described below by way of example only.

It will be understood that the benefits and advantages described herein may relate to one embodiment or may relate to several embodiments. The embodiments are not limited to those that solve any or all of the stated problems or those that have any or all of the stated benefits and advantages.

Features of different aspects and embodiments may be combined as appropriate, as would be apparent to a skilled person, and may be combined with any of the aspects as described herein. Advantageous and/or exemplary features of the constructs are as follows:

1) they contain multiple (2 or more) at least partially double-stranded agents that trigger RNA interference, tied together into a single nano-structure predominantly through complementary (Watson-Crick) interactions;

2) optionally, other (e.g.) covalent bindings may be used to build the constructs and/or add various ligands (e.g., delivery/targeting moieties such as GalNAc and or other carbohydrates, cholesterol, peptides, or small molecules, optionally attached via linkers);

3) the constructs predominantly comprise chemically modified nucleotides (e.g., 2′F, 2′OMe, LNO, PNA, MOE, BNA, PMO, phosphorothioate, phosphodithioate, etc. etc), mostly (but not only) to increase resistance to nucleases;

4) the constructs contain “fragile” components (e.g., chemical linkers, unmodified nucleotides, etc), which allow the constructs to disassemble upon exposure to certain biologic environments (e.g., exposure to extra- and/or intra-cellular fluids); specific examples include, but are not limited to: a) cleavage of the oligo backbone by nucleases in the sites with non-modified nucleotides; b) cleavage of the chemical linkage due to the change of pH (e.g., in endosomes);

5) disassembly upon exposure to said certain biologic environments releases the active components (e.g., said at least partially double-stranded agents that trigger RNA interference) to modulate (up- or down-regulate, optionally down-regulate) target gene expression in cells/organisms;

6) said constructs can be used to modulate, optionally down-regulate or silence gene expression, to study gene function, or to treat various diseases associated with the target genes to be down-regulated.

### Definitions

Unless specific definitions are provided, the nomenclature used in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21^{st} edition, 2005; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2^{nd} Edition, Cold Spring Harbor Laboratory Press, 1989, which are hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

As used herein, “excipient” means any compound or mixture of compounds that is added to a composition as provided herein that is suitable for delivery of an oligomeric compound.

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety, phosphate-linked nucleosides also being referred to as “nucleotides”.

As used herein, “chemical modification” or “chemically modified” means a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

As used herein, “furanosyl” means a structure comprising a 5-membered ring comprising four carbon atoms and one oxygen atom.

As used herein, “naturally occurring sugar moiety” means a ribofuranosyl as found in naturally occurring RNA or a deoxyribofuranosyl as found in naturally occurring DNA. A “naturally occurring sugar moiety” as referred to herein is also termed as an “unmodified sugar moiety”.

A “naturally occurring sugar moiety” or an “unmodified sugar moiety” as referred to herein has a —H (DNA sugar moiety) or —OH (RNA sugar moiety) at the 2′-position of the sugar moiety, especially a —H (DNA sugar moiety) at the 2′-position of the sugar moiety.

As used herein, “sugar moiety” means a naturally occurring sugar moiety or a modified sugar moiety of a nucleoside. As used herein, “modified sugar moiety” means a substituted sugar moiety or a sugar surrogate.

As used herein, “substituted sugar moiety” means a furanosyl that has been substituted.

Substituted sugar moieties include, but are not limited to furanosyls comprising substituents at the 2′-position, the 3′-position, the 5′-position and/or the 4′-position. Certain substituted sugar moieties are bicyclic sugar moieties.

As used herein, “2′-substituted sugar moiety” means a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring).

As used herein, “MOE” means —OCH₂CH₂OCH₃.

As used herein, “2′-F nucleoside” refers to a nucleoside comprising a sugar comprising fluorine at the 2′ position. Unless otherwise indicated, the fluorine in a 2′-F nucleoside is in the ribo position (replacing the OH of a natural ribose). Duplexes of uniformly modified 2′-fluorinated (ribo) oligonucleotides hybridized to RNA strands are not RNase H substrates while the ara analogs retain RNase H activity.

As used herein the term “sugar surrogate” means a structure that does not comprise a furanosyl and that replaces the naturally occurring sugar moiety of a nucleoside, such that the resulting nucleoside sub-units are capable of linking together and/or linking to other nucleosides to form an oligomeric compound which hybridizes to a complementary oligomeric compound. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols.

As used herein, “bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2 ‘-carbon and the 4’-carbon of the furanosyl.

As used herein, “nucleotide” means a nucleoside further comprising a phosphate linking group. As used herein, “linked nucleosides” may or may not be linked by phosphate linkages and thus includes, but is not limited to “linked nucleotides.” As used herein, “linked nucleosides” are nucleosides that are connected in a continuous sequence (i.e., no additional nucleosides are present between those that are linked).

As used herein, “nucleobase” means a group of atoms that can be linked to a sugar moiety to create a nucleoside that may be incorporated into an oligonucleotide, and where the group of atoms is capable of bonding, more specifically hydrogen bonding, with a complementary naturally occurring nucleobase of another oligonucleotide or nucleic acid. Nucleobases may be naturally occurring or may be modified.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase.

As used herein, “modified nucleoside” means a nucleoside comprising at least one chemical modification compared to naturally occurring RNA or DNA nucleosides. Modified nucleosides can comprise a modified sugar moiety and/or a modified nucleobase.

As used herein, “bicyclic nucleoside” or “BNA” means a nucleoside comprising a bicyclic sugar moiety.

As used herein, “locked nucleic acid nucleoside” or “LNA” means a nucleoside comprising a bicyclic sugar moiety comprising a 4′-CH₂—O-2′bridge.

As used herein, “2 ‘-substituted nucleoside” means a nucleoside comprising a substituent at the 2’-position of the sugar moiety other than H or OH. Unless otherwise indicated, a 2′-substituted nucleoside is not a bicyclic nucleoside.

As used herein, “deoxynucleoside” means a nucleoside comprising 2′-H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA) and/or one or more modified nucleosides.

As used herein, “modified oligonucleotide” means an oligonucleotide comprising at least one modified nucleoside and/or at least one modified internucleoside linkage.

As used herein, “linkage” or “linking group” means a group of atoms that link together two or more other groups of atoms.

As used herein “internucleoside linkage” means a covalent linkage between adjacent nucleosides in an oligonucleotide.

As used herein “naturally occurring internucleoside linkage” means a 3′ to 5′ phosphodiester linkage. As used herein, “modified internucleoside linkage” means any internucleoside linkage other than a naturally occurring internucleoside linkage. A “modified internucleoside linkage” as referred to herein can include a modified phosphorous linking group such as a phosphorothioate or phosphorodithioate internucleoside linkage.

As used herein, “terminal internucleoside linkage” means the linkage between the last two nucleosides of an oligonucleotide or defined region thereof.

As used herein, “phosphorus linking group” means a linking group comprising a phosphorus atom and can include naturally occurring phosphorous linking groups as present in naturally occurring RNA or DNA, such as phosphodiester linking groups, or modified phosphorous linking groups that are not generally present in naturally occurring RNA or DNA, such as phosphorothioate or phosphorodithioate linking groups. Phosphorus linking groups can therefore include without limitation, phosphodiester, phosphorothioate, phosphorodithioate, phosphonate, methylphosphonate, phosphoramidate, phosphorothioamidate, thionoalkylphosphonate, phosphotriesters, thionoalkylphosphotriester and boranophosphate.

As used herein, “internucleoside phosphorus linking group” means a phosphorus linking group that directly links two nucleosides.

As used herein, “oligomeric compound” means a polymeric structure comprising two or more substructures. In certain embodiments, an oligomeric compound comprises an oligonucleotide, such as a modified oligonucleotide. In certain embodiments, an oligomeric compound further comprises one or more conjugate groups and/or terminal groups and/or ligands. In certain embodiments, an oligomeric compound consists of an oligonucleotide. In certain embodiments, an oligomeric compound comprises a backbone of one or more linked monomeric sugar moieties, where each linked monomeric sugar moiety is directly or indirectly attached to a heterocyclic base moiety. In certain embodiments, oligomeric compounds may also include monomeric sugar moieties that are not linked to a heterocyclic base moiety, thereby providing abasic sites. Oligomeric compounds may be defined in terms of a nucleobase sequence only, i.e., by specifying the sequence of A, G, C, U (or T). In such a case, the structure of the sugar-phosphate backbone is not particularly limited and may or may not comprise modified sugars and/or modified phosphates. On the other hand, oligomeric compounds may be more comprehensively defined, i.e., by specifying not only the nucleobase sequence, but also the structure of the backbone, including the modification status of the sugars (unmodified, 2′-OMe modified, 2′-F modified etc.) and/or of the phosphates.

As used herein, “nucleic acid construct” or “construct” refers to an assembly of two or more, such as four oligomeric compounds. The oligomeric compounds may be connected to each other by covalent bonds such phosphodiester bonds as they occur in naturally occurring nucleic acids or modified versions thereof as disclosed herein, or by non-covalent bonds such as hydrogen bonds, optionally hydrogen bonds between nucleobases such as Watson-Crick base pairing. Advantageously, a construct comprises four oligomeric compounds, two of which are connected covalently, thereby giving rise to two nucleic acid strands which nucleic acid strands are bound to each other by hydrogen bonds. Complementarity between the strands may be throughout, but is not necessarily so. Specific embodiments provide for an antisense strand targeting PCSK9 to be connected covalently with a sense strand of an APOC3-targeting double stranded RNA molecule, and of the antisense strand of the APOC3-targeting double stranded RNA molecule to be connected covalently to a sense strand of a PCSK9-targeting double stranded RNA molecule. Since antisense and sense strands of the parent single-target-directed RNA molecules do not need to have the same length and optionally do not have the same length with antisense portions being longer than sense portions, advantageously the construct contains a central region where the 3′ regions of the antisense portions of the parent single-target-directed RNA molecules face each other. In that region generally no or only partial base pairing will occur, while full complementarity is not excluded. Otherwise, where antisense and sense portions of the respective parent RNA molecules face each other, there is complementarity, optionally full complementarity or 1 or 2 mismatches.

The term “strand” has its art-established meaning and refers to a plurality of linked nucleosides, the linker not being particularly limited, but including phosphodiesters and variants thereof as disclosed herein. A strand may also be viewed as a plurality of linked nucleotides in which case the linker would be a covalent bond.

As used herein, “terminal group” means one or more atom attached to either, or both, the 3′ end or the 5′ end of an oligonucleotide. In certain embodiments, a terminal group comprises one or more terminal group nucleosides.

As used herein, “conjugate” or “conjugate group” means an atom or group of atoms bound to an oligonucleotide or oligomeric compound. In certain embodiments, a conjugate group links a ligand to a modified oligonucleotide or oligomeric compound. In general, conjugate groups can modify one or more properties of the compound to which they are attached, including, but not limited to pharmacodynamic, pharmacokinetic, binding, absorption, cellular distribution, cellular uptake, charge and/or clearance properties.

As used herein, “conjugate linker” or “linker” in the context of a conjugate group means a portion of a conjugate group comprising any atom or group of atoms and which covalently link an oligonucleotide to another portion of the conjugate group. In certain embodiments, the point of attachment on the oligomeric compound is the 3 ‘-oxygen atom of the 3’-hydroxyl group of the 3′ terminal nucleoside of the oligonucleotide. In certain embodiments the point of attachment on the oligomeric compound is the 5′-oxygen atom of the 5′-hydroxyl group of the 5′ terminal nucleoside of the oligonucleotide. In certain embodiments, the bond for forming attachment to the oligomeric compound is a cleavable bond. In certain such embodiments, such cleavable bond constitutes all or part of a cleavable moiety.

In certain embodiments, conjugate groups comprise a cleavable moiety (e.g., a cleavable bond or cleavable nucleoside) and ligand portion that can comprise one or more ligands, such as a carbohydrate cluster portion, such as an N-Acetyl-Galactosamine, also referred to as “GalNAc”, cluster portion. In certain embodiments, the carbohydrate cluster portion is identified by the number and identity of the ligand. For example, in certain embodiments, the carbohydrate cluster portion comprises 2 GalNAc groups. For example, in certain embodiments, the carbohydrate cluster portion comprises 3 GalNAc groups. In certain embodiments, the carbohydrate cluster portion comprises 4 GalNAc groups. Such ligand portions are attached to an oligomeric compound via a cleavable moiety, such as a cleavable bond or cleavable nucleoside. The ligands can be arranged in a linear or branched configuration, such as a biantennary or triantennary configurations. An optional carbohydrate cluster has the following formula:

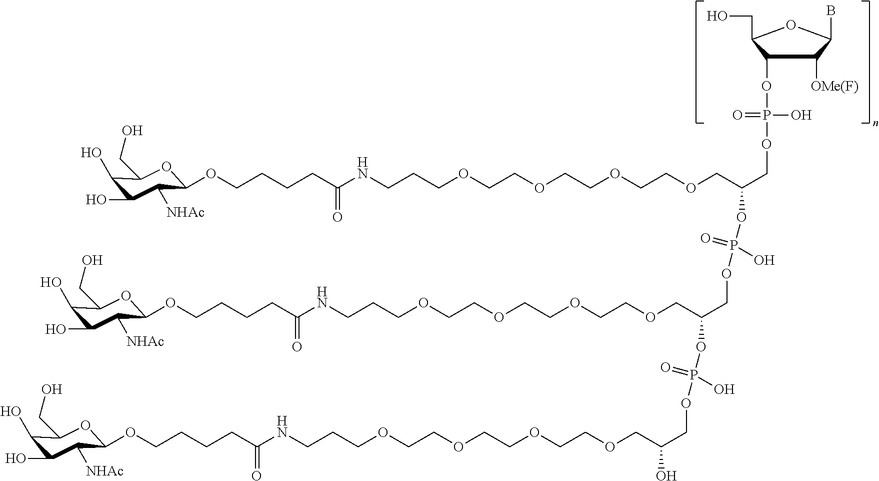

wherein in said structural formula one, two, or three phosphodiester linkages can also be substituted by phosphorothioate linkages.

As used herein, “cleavable moiety” means a bond or group that is cleaved under physiological conditions. In certain embodiments, a cleavable moiety is cleaved inside a cell or sub-cellular compartments, such as an endosome or lysosome. In certain embodiments, a cleavable moiety is cleaved by endogenous enzymes, such as nucleases. In certain embodiments, a cleavable moiety comprises a group of atoms having one, two, three, four, or more than four cleavable bonds. In certain embodiments, a cleavable moiety is a phosphodiester linkage.

As used herein, “cleavable bond” means any chemical bond capable of being broken.

As used herein, “carbohydrate cluster” means a compound having one or more carbohydrate residues attached to a linker group.

As used herein, “modified carbohydrate” means any carbohydrate having one or more chemical modifications relative to naturally occurring carbohydrates.

As used herein, “carbohydrate derivative” means any compound which may be synthesized using a carbohydrate as a starting material or intermediate.

As used herein, “carbohydrate” means a naturally occurring carbohydrate, a modified carbohydrate, or a carbohydrate derivative. A carbohydrate is a biomolecule including carbon (C), hydrogen (H) and oxygen (O) atoms. Carbohydrates can include monosaccharide, disaccharides, trisaccharides, tetrasaccharides, oligosaccharides or polysaccharides, such as one or more galactose moieties, one or more lactose moieties, one or more N-Acetyl-Galactosamine moieties, and/or one or more mannose moieties. Advantageously, the carbohydrate is N-Acetyl-Galactosamine.

As used herein, “strand” means an oligomeric compound comprising linked nucleosides.

As used herein, “single strand” or “single-stranded” means an oligomeric compound comprising linked nucleosides that are connected in a continuous sequence without a break there between. Such single strands may include regions of sufficient self-complementarity so as to be form a stable self-duplex in a hairpin structure.

As used herein, “hairpin” means a single stranded oligomeric compound that includes a duplex formed by base pairing between sequences in the strand that are self-complementary and opposite in directionality.

As used herein, “hairpin loop” means an unpaired loop of linked nucleosides in a hairpin that is created as a result of hybridization of the self-complementary sequences. The resulting structure looks like a loop or a U-shape.

As used herein, “directionality” means the end-to-end chemical orientation of an oligonucleotide based on the chemical convention of numbering of carbon atoms in the sugar moiety meaning that there will be a 5′-end defined by the 5′ carbon of the sugar moiety, and a 3′-end defined by the 3′ carbon of the sugar moiety. In a duplex or double stranded oligonucleotide, the respective strands run in opposite 5′ to 3′ directions to permit base pairing between them.

As used herein, “duplex” means two or more complementary strand regions, or strands, of an oligonucleotide or oligonucleotides, hybridized together by way of non-covalent, sequence-specific interaction therebetween. Most commonly, the hybridization in the duplex will be between nucleobases adenine (A) and thymine (T), and/or (A) adenine and uracil (U), and/or guanine (G) and cytosine (C). The duplex may be part of a single stranded structure, wherein self-complementarity leads to hybridization, or as a result of hybridization between respective strands in a double stranded construct.

As used herein, “double strand” or “double stranded” means a pair of oligomeric compounds that are hybridized to one another. In certain embodiments, a double-stranded oligomeric compound comprises a first and a second oligomeric compound.

As used herein, “expression” means the process by which a gene ultimately results in a protein. Expression includes, but is not limited to, transcription, post-transcriptional modification (e.g., splicing, polyadenlyation, addition of 5′-cap), and translation.

As used herein, “transcription” or “transcribed” refers to the first of several steps of DNA based gene expression in which a target sequence of DNA is copied into RNA (especially mRNA) by the enzyme RNA polymerase. During transcription, a DNA sequence is read by an RNA polymerase, which produces a complementary, antiparallel RNA sequence called a primary transcript.

As used herein, “target sequence” means a sequence to which an oligomeric compound is intended to hybridize to result in a desired activity with respect to PCSK9 and/or APOC3 expression. Oligonucleotides have sufficient complementarity to their target sequences to allow hybridization under physiological conditions.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In both DNA and RNA, guanine (G) is complementary to cytosine (C). In certain embodiments, complementary nucleobase means a nucleobase of an oligomeric compound that is capable of base pairing with a nucleobase of its target sequence. For example, if a nucleobase at a certain position of an oligomeric compound is capable of hydrogen bonding with a nucleobase at a certain position of a target sequence, then the position of hydrogen bonding between the oligomeric compound and the target sequence is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another.

As used herein, “complementary” in reference to oligomeric compounds (e.g., linked nucleosides, oligonucleotides) means the capacity of such oligomeric compounds or regions thereof to hybridize to a target sequence, or to a region of the oligomeric compound itself, through nucleobase complementarity.

Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or regions are 80%>complementary. In certain embodiments, complementary oligomeric compounds or regions are 90%>complementary. In certain embodiments, complementary oligomeric compounds or regions are at least 95% complementary. In certain embodiments, complementary oligomeric compounds or regions are 100% complementary.

As used herein, “self-complementarity” in reference to oligomeric compounds means a compound that may fold back on itself, creating a duplex as a result of nucleobase hybridization of internal complementary strand regions. Depending on how close together and/or how long the strand regions are, then the compound may form hairpin loops, junctions, bulges or internal loops.

As used herein, “mismatch” means a nucleobase of an oligomeric compound that is not capable of pairing with a nucleobase at a corresponding position of a target sequence, or at a corresponding position of the oligomeric compound itself when the oligomeric compound hybridizes as a result of self-complementarity, when the oligomeric compound and the target sequence and/or self-complementary regions of the oligomeric compound, are aligned.

As used herein, “hybridization” means the pairing of complementary oligomeric compounds (e.g., an oligomeric compound and its target sequence). While not limited to a particular mechanism, the most common mechanism of pairing involves hydrogen bonding, which may be Watson-Crick, Hoogsteen or reversed Hoogsteen hydrogen bonding, between complementary nucleobases.

As used herein, “specifically hybridizes” means the ability of an oligomeric compound to hybridize to one nucleic acid site with greater affinity than it hybridizes to another nucleic acid site.

As used herein, “fully complementary” in reference to an oligomeric compound or region thereof means that each nucleobase of the oligomeric compound or region thereof is capable of pairing with a nucleobase of a complementary nucleic acid target sequence or a self-complementary region of the oligomeric compound. Thus, a fully complementary oligomeric compound or region thereof comprises no mismatches or unhybridized nucleobases with respect to its target sequence or a self-complementary region of the oligomeric compound.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein, “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression.

As used herein, “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

As used herein, “differently modified” mean chemical modifications or chemical substituents that are different from one another, including absence of modifications. Thus, for example, a MOE nucleoside and an unmodified naturally occurring RNA nucleoside are “differently modified,” even though the naturally occurring nucleoside is unmodified. Likewise, DNA and RNA oligonucleotides are “differently modified,” even though both are naturally-occurring unmodified nucleosides. Nucleosides that are the same but for comprising different nucleobases are not differently modified. For example, a nucleoside comprising a 2′-OMe modified sugar moiety and an unmodified adenine nucleobase and a nucleoside comprising a 2′-OMe modified sugar moiety and an unmodified thymine nucleobase are not differently modified.

As used herein, “the same type of modifications” refers to modifications that are the same as one another, including absence of modifications. Thus, for example, two unmodified RNA nucleosides have “the same type of modification,” even though the RNA nucleosides are unmodified. Such nucleosides having the same type modification may comprise different nucleobases.

As used herein, “region” or “regions”, or “portion” or “portions”, mean a plurality of linked nucleosides that have a function or character as defined herein, in particular with reference to the claims and definitions as provided herein. Typically such regions or portions comprise at least 10, at least 11, at least 12 or at least 13 linked nucleosides. For example, such regions can comprise 13 to 20 linked nucleosides, such as 13 to 16 or 18 to 20 linked nucleosides.

Typically a first region as defined herein consists essentially of 18 to 20 nucleosides and a second region as defined herein consists essentially of 13 to 16 linked nucleosides.

As used herein, “pharmaceutically acceptable carrier or diluent” means any substance suitable for use in administering to an animal. In certain embodiments, a pharmaceutically acceptable carrier or diluent is sterile saline. In certain embodiments, such sterile saline is pharmaceutical grade saline.

As used herein, “substituent” and “substituent group,” means an atom or group that replaces the atom or group of a named parent compound. For example a substituent of a modified nucleoside is any atom or group that differs from the atom or group found in a naturally occurring nucleoside (e.g., a modified 2′-substituent is any atom or group at the 2′-position of a nucleoside other than H or OH). Substituent groups can be protected or unprotected. In certain embodiments, compounds of the present disclosure have substituents at one or at more than one position of the parent compound. Substituents may also be further substituted with other substituent groups and may be attached directly or via a linking group such as oxygen or an alkyl or hydrocarbyl group to a parent compound.

Such substituents can be present as the modification on the sugar moiety, for example a substituent present at the 2′-position of the sugar moiety. Unless otherwise indicated, groups amenable for use as substituents include without limitation, one or more of halo, hydroxyl, alkyl, alkenyl, alkynyl, acyl, carboxyl, alkoxy, alkoxyalkylene and amino substituents. Certain substituents as described herein can represent modifications directly attached to a ring of a sugar moiety (such as a halo, such as fluoro, directly attached to a sugar ring), or a modification indirectly linked to a ring of a sugar moiety by way of an oxygen linking atom that itself is directly linked to the sugar moiety (such as an alkoxyalkylene, such as methoxyethylene, linked to an oxygen atom, overall providing an MOE substituent as described herein attached to the 2′-position of the sugar moiety).

As used herein, “alkyl,” as used herein, means a saturated straight or branched monovalent C₁₋₆ hydrocarbon radical. Advantageously, methyl is the alkyl substituent at the 2′-position of the sugar moiety. The alkyl group typically attaches to an oxygen linking atom at the 2′position of the sugar, therefore, overall providing an —O-alkyl substituent, such as an —OCH₃ substituent, on a sugar moiety of an oligomeric compound as described herein. This will be well understood be a person skilled in the art.

As used herein, “alkylene” means a saturated straight or branched divalent hydrocarbon radical of the general formula —CₙH₂ₙ— where n is 1-6. Methylene or ethylene are examples of alkylenes.

As used herein, “alkenyl” means a straight or branched unsaturated monovalent C₂₋₆ hydrocarbon radical. Ethenyl or propenyl moieties are examples of alkenyls as a substituent at the 2′-position of the sugar moiety. As will be well understood in the art, the degree of unsaturation that is present in an alkenyl radical is the presence of at least one carbon to carbon double bond. The alkenyl group typically attaches to an oxygen linking atom at the 2′-position of the sugar, therefore, overall providing a —Oalkenyl substituent, such as an —OCH₂CH═CH₂ substituent, on a sugar moiety of an oligomeric compound a as described herein. This will be well understood be a person skilled in the art.

As used herein, “alkynyl” means a straight or branched unsaturated C₂₋₆ hydrocarbon radical. Ethynyl is an example of an alkynyl as a substituent at the 2′-position of the sugar moiety. As will be well understood in the art, the degree of unsaturation that is present in an alkynyl radical is the presence of at least one carbon to carbon triple bond. The alkynyl group typically attaches to an oxygen linking atom at the 2′-position of the sugar, therefore, overall providing an —O-alkynyl substituent on a sugar moiety of an oligomeric compound as described herein. This will be well understood be a person skilled in the art.

As used herein, “carboxyl” is a radical having a general formula —CO₂H.

As used herein, “acyl” means a radical formed by removal of a hydroxyl group from a carboxyl radical as defined herein and has the general Formula —C(O)—X where X is typically C₁₋₆ alkyl.

As used herein, “alkoxy” means a radical formed between an alkyl group, such as a C₁₋₆ alkyl group, and an oxygen atom wherein the oxygen atom is used to attach the alkoxy group either to a parent molecule (such as at the 2′-position of a sugar moiety), or to another group such as an alkylene group as defined herein. Examples of alkoxy groups include without limitation, methoxy, ethoxy, propoxy, isopropoxy, n-butoxy, sec-butoxy and tert-butoxy. Alkoxy groups as used herein may optionally include further substituent groups.

As used herein, alkoxyalkylene means an alkoxy group as defined herein that is attached to an alkylene group also as defined herein, and where the oxygen atom of the alkoxy group attaches to the alkylene group and the alkylene attaches to a parent molecule. The alkylene group typically attaches to an oxygen linking atom at the 2′-position of the sugar, therefore, overall providing a —Oalkylenealkoxy substituent, such as an —OCH₂CH₂OCH₃ substituent, on a sugar moiety of an oligomeric compound as described herein. This will be well understood by a person skilled in the art and is generally referred to as an MOE substituent as defined herein and as known in the art.

As used herein, “amino” includes primary, secondary and tertiary amino groups.

As used herein, “halo” and “halogen,” mean an atom selected from fluorine, chlorine, bromine and iodine.

As used herein, the term “muRNA” or “multi RNA” may include nucleic acid constructs comprising more than one, typically two, RNA sequences, i.e., first and second nucleic acid portions, targeting one region of PCSK9 mRNA and an mRNA region of APOC3. The targeting RNA sequences are also referred to as “antisense” or “guide” strands, while the respective passenger strands, i.e., third and fourth nucleic acid portions being complementary to the first and second portion, respectively, are also included in the nucleic acid construct. In particular, such muRNA are designed such that subsequent to in vivo administration, they are disassembled and said first and second nucleic acid portions are released. A particular example for such muRNA is shown below, where (1) is the first nucleic acid portion, (2) is the third nucleic acid portion being complementary to (1), (3) is the second nucleic acid portion being complementary to the fourth nucleic acid portion, while (5) is a labile linker while (6) is a ligand, which will both be explained below.

Further miniaturization by shortening the sense regions leads to bulge in the central part of the molecule where the 3′-terminal regions of the two antisense regions face each other:

In the diagram above, “GN” designates a GalNAc moiety, and “SBS” designates the fragile site which may be implemented as a nucleoside with a non-modified sugar.

As used herein “PC1” and “PC1a” are two PCSK9 siRNA sequences that have the same siRNA sequences with different GalNAc conjugations. These compounds are known to be effective in PCSK9 gene knockdown and were used as positive control compounds in the Examples section.

It will also be understood that oligomeric compounds as described herein may have one or more non-hybridizing nucleosides at one or both ends of one or both strands (overhangs) and/or one or more internal non-hybridizing nucleosides (mismatches) provided there is sufficient complementarity to maintain hybridization under physiologically relevant conditions.

Alternatively, oligomeric compounds as described herein may be blunt ended at at least one end.

The term “comprising” is used herein to mean including the method steps or elements identified, but that such steps or elements do not comprise an exclusive list and as such there may be present additional steps or elements.

Further, to the extent that the term “includes” is used in either the detailed description or the claims, such term is intended to be inclusive in a manner similar to the term “comprising” as “comprising” is interpreted when employed as a transitional word in a claim.

The Following Embodiments are Provided (Items):

1. A nucleic acid construct comprising at least:

(a) a first nucleic acid portion that is at least partially complementary to at least a first portion of an RNA which is transcribed from a PCSK9 gene;

(b) a second nucleic acid portion that is at least partially complementary to at least a second portion of an RNA which is transcribed from a APOC3 gene;

(c) a third nucleic acid portion that is at least partially complementary to said first nucleic acid portion of (a), so as to form a first nucleic acid duplex region therewith;

(d) a fourth nucleic acid portion that is at least partially complementary to said second nucleic acid portion of (b), so as to form a second nucleic acid duplex region therewith.

2. The construct according to item 1, wherein said construct is designed such that subsequent to in vivo administration said construct disassembles to yield at least first and second discrete nucleic acid targeting molecules that respectively target said RNA portions transcribed from said target genes of (a) and (b);

whereby (i) said first nucleic acid targeting molecule modulates expression of said target gene of (a), and comprises, or is derived from, at least said first nucleic acid portion of (a), and (ii) said second nucleic acid targeting molecule modulates expression of said target gene of (b), and comprises, or is derived from, said second nucleic acid portion of (b).

3. The construct according to item 1 or 2, wherein said construct is designed to disassemble such that said first and second discrete nucleic acid targeting molecules are respectively processed by independent RNAi-induced silencing complexes.

4. The construct according to any one of items 1 to 3, which further comprises at least one labile functionality such that subsequent to in vivo administration said construct is cleaved so as to yield said at least first and second discrete nucleic acid targeting molecules.

5. The construct according to item 4, wherein said labile functionality comprises one or more unmodified nucleotides.

6. The construct according to item 5, wherein said one or more unmodified nucleotides of said labile functionality represent one or more cleavage positions within said construct whereby subsequent to in vivo administration said construct is cleaved at said one or more cleavage positions so as to yield said at least first and second discrete nucleic acid targeting molecules.

7. The construct according to item 6, wherein said cleavage positions are respectively located within the construct so that subsequent to cleavage said first discrete nucleic acid targeting molecule comprises, or is derived from, said first nucleic acid duplex region, and said second discrete nucleic acid targeting molecule comprises, or is derived from, said second nucleic acid duplex region.

8. The construct according to item 7, wherein said first discrete nucleic acid targeting molecule comprises or consists of said first nucleic acid portion of (a) and said third nucleic acid portion of (c), and/or said second discrete nucleic acid targeting molecule comprises or consists of said second nucleic acid portion of (b) and said fourth nucleic acid portion of (d).

9. The construct according to any one of items 1 to 8, wherein

(a) said first nucleic acid portion has a nucleobase sequence selected from Table 1a (SEQ ID NOs: 1 to 7) or SEQ ID NO: 31;

(b) said second nucleic acid portion has a nucleobase sequence selected from Table 1b (SEQ ID NOs: 8 to 14) or SEQ ID NO: 29;

(c) said third nucleic acid portion has a nucleobase sequence selected from Table 2a (SEQ ID NOs: 15 to 21) or SEQ ID NO: 32; and/or

(d) said fourth nucleic acid portion has a nucleobase sequence selected from Table 2b (SEQ ID NOs: 22 to 28) or SEQ ID NO: 30.

10. The construct according to any of items 1 to 9, wherein said first nucleic acid portion of

(a) is directly or indirectly linked to said fourth nucleic acid portion of (d) as a primary structure.

11. The construct according to item 10, wherein said first and said fourth nucleic acid portions have the nucleobase sequences of SEQ ID NOs: 2 and 22; 4 and 24; 5 and 22; 6 and 24; 4 and 22; 6 and 22; 31 and 24; 31 and 22; 4 and 25; 5 and 26; 3 and 28; or 1 and 30, respectively.

12. The construct according to any of items 1 to 11, wherein said second nucleic acid portion of (b) is directly or indirectly linked to said third nucleic acid portion of (c) as a primary structure.

13. The construct according to item 11 or 12, wherein said second and third nucleic acid portions have the nucleobase sequences of SEQ ID NOs: 8 and 16; 10 and 18; 8 and 19; 10 and 20; 8 and 18; 8 and 20; 10 and 32; 8 and 32; 11 and 18; 12 and 19; 14 and 17; or 29 and 15, respectively.

14. The construct according to any of items 1 to 9, 11 or 13, that further comprises 1 to 8 additional nucleic acid portions that are respectively at least partially complementary to an additional 1 to 8 portions of RNA transcribed from one or more target genes, which target genes may be the same or different to each other, and/or the same or different to the target genes defined in (a) and/or (b), and wherein each of the 1 to 8 additional nucleic acid portions respectively form additional duplex regions with respective passenger nucleic acid portions that are respectively at least partially complementary therewith.

15. The construct according to item 14, wherein said second nucleic acid portion of (b), and said 1 to 8 additional nucleic acid portions, are directly or indirectly linked to selected passenger nucleic acid portions as respective primary structures.

16. The construct according to any of items 10, 12 or 15, wherein said direct or indirect linking represents either (i) an internucleotide bond, (ii) an internucleotide nick, or (iii) a nucleic acid linker portion of 1, 2, 3, 4, 5, 6, 7, 8, 9, or 10 nucleotides, said nucleic acid linker optionally being single stranded.

17. The construct according to item 16 (i), wherein said linking is direct, thereby giving rise to (a) contiguous strand(s).

18. The construct of any one of items 1 to 17, especially of item 16 (i), wherein there exists some complementarity between the first nucleic acid portion of (a) and the second nucleic acid portion of (b), or the third nucleic acid portion of (c) and the fourth nucleic acid portion of (d).

19. The construct according to item 18, wherein said complementarity

(i) is/are 1, 2, 3, 4, 5, 6, 7, 8, 9, or 10, optionally 2, 3, 4 or 5 base pairs; and/or

(ii) is between the first nucleic acid portion of (a) and the second nucleic acid portion of (b).

20. The construct according to item 16 (i) to 19, as dependent on item 5, wherein said internucleotide bond involves at least one of said one or more unmodified nucleotides, wherein optionally cleavage occurs at the 3′ position of (at least one of) said unmodified nucleotide(s).

21. The construct according to any of items 1 to 20, wherein said first nucleic acid portion of (a), and/or said second nucleic acid portion of (b), and/or said third nucleic acid portion of (c), and/or said fourth nucleic acid portion of (d), are respectively 7 to 25 nucleotides in length.

22. The construct according to item 21, wherein said first nucleic acid portion of (a) and/or said second nucleic acid portion of (b) have a length of 18 to 21, more optionally 18 or 19, and yet more optionally 19 nucleotides.

23. The construct according to item 21 or 22, wherein said third nucleic acid portion of (c), and/or said fourth nucleic acid portion of (d) have a length of 11 to 20, more optionally 13 to 16, and yet more optionally 14 or 15, most optionally 14 nucleotides.

24. The construct according to any one of items 21 to 23, wherein said unmodified nucleotide(s) is/are at any of position 18 to 25, more optionally at any of positions 18 to 21, and most optionally at position 19 and/or the 3′ terminal position of said first nucleic acid portion of (a) and/or of said third nucleic acid portion of (c).

26. The construct according to any of items 17 to 19 or 21 to 23 as dependent on item 16(iii), wherein said nucleic acid linker portion is 1 to 8 nucleotides in length, optionally 2 to 7 or 3 to 6 nucleotides in length, more optionally about 4 or 5 and most optionally 4 nucleotides in length.

27. The construct according to any one of items 21 to 26, wherein one, more of all of the duplex regions independently have a length of 11 to 19, more optionally 14 to 19, and yet more optionally 14 or 15 base pairs, most optionally 14 base pairs, wherein optionally there is one mismatch within said duplex region.

28. The construct according to any of items 1 to 27, which further comprises one or more ligands.

29. The construct according to any one of items 1 to 28, wherein said first nucleic acid portion of (a), and/or said second nucleic acid portion of (b), and/or said third nucleic acid portion of (c), and/or said fourth nucleic acid portion of (d), and/or, to the extent present, said 1 to 8 additional nucleic acid portions as defined in items 14 and 15, and/or said passenger nucleic acid portions as defined in items 14 or 15, respectively have a 5′ to 3′ directionality thereby defining 5′ and 3′ regions thereof.

30. The construct according to any one of items 28 or 29, wherein one or more ligands are conjugated at the 3′ region, optionally the 3′ end, of any of (i) said third nucleic acid portion of (c), and/or (ii) said fourth nucleic acid portion of (d), and/or, to the extent present, said (iii) passenger nucleic acid portions as defined in items 14 or 15.

31. The construct according to any one of items 28 to 30, wherein one or more ligands are conjugated at one or more regions intermediate of the 5′ and 3′ regions of any of said nucleic acid portions, optionally of said third nucleic acid portion of (c), and/or said fourth nucleic acid portion of (d), and/or said passenger nucleic acid portions as defined in items 14 or 15.

32. The construct of any one of items 28 to 31, wherein one or more ligands are conjugated at the 5′ region, optionally the 5′ end, of any of said nucleic acid portions.

33. The construct according to any of items 28 to 32, wherein said one or more ligands are any cell directing moiety, such as lipids, carbohydrates, aptamers, vitamins and/or peptides that bind cellular membrane or a specific target on cellular surface.

34. The construct according to item 33, wherein said one or more carbohydrates can be a monosaccharide, disaccharide, trisaccharide, tetrasaccharide, oligosaccharide or polysaccharide.

35. The construct according to item 34, wherein said one or more carbohydrates comprise one or more hexose moieties.

36. The construct of item 35, wherein said one or more hexose moieties are one or more galactose moieties, one or more lactose moieties, one or more N-Acetyl-Galactosamine moieties, and/or one or more mannose moieties.

37. The construct according to item 36, which comprises two or three N-Acetyl-Galactosamine moieties.

38. The construct according to any of items 28 to 37, wherein said one or more ligands are attached in a linear configuration, or in a branched configuration.

39. The construct according to item 38, wherein said one or more ligands are attached as a biantennary or triantennary configuration, or as a configuration based on single ligands at different positions.

40. The construct according to item 37 or 38, wherein said ligand has the following structure:

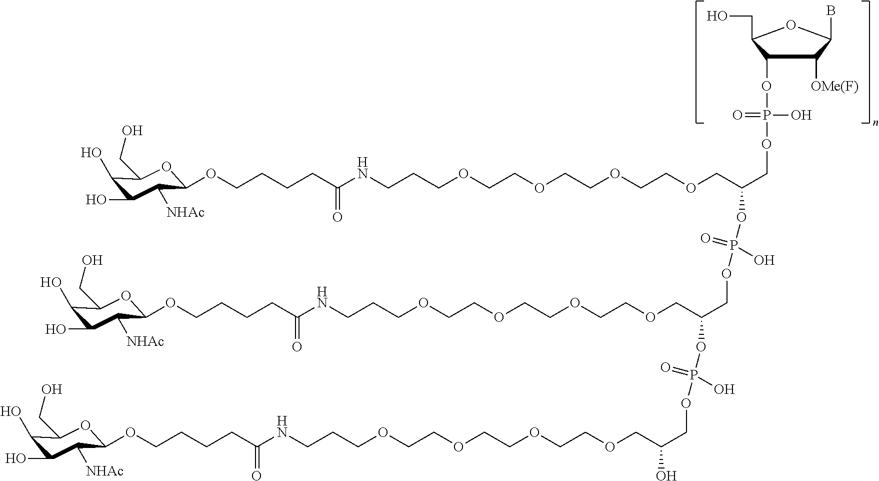

41. The construct according to any of items 1 to 40, which further comprises one or more phosphorothioate or phosphorodithioate internucleotide linkages.

42. The construct according item 41, which comprises 1 to 15 phosphorothioate or phosphorodithioate internucleotide linkages.

43. The construct according to item 41 or 42, which comprises one or more phosphorothioate or phosphorodithioate internucleotide linkages at one or more of the 5′ and/or 3′ regions of said first nucleic acid portion of (a), and/or said second nucleic acid portion of (b), and/or said third nucleic acid portion of (c), and/or said fourth nucleic acid portion of (d), and/or said 1 to 8 additional nucleic acid portions as defined in items 14 or 15, and/or said passenger nucleic acid portions as defined in items 14 or 15.

44. The construct according to any of items 41 to 43, which comprises phosphorothioate or phosphorodithioate internucleotide linkages between at least two adjacent nucleotides of the nucleic acid linker portion as defined in item 16 (iii).

45. The construct according to any of item 44, which comprises a phosphorothioate or phosphorodithioate internucleotide linkage between each adjacent nucleotide that is present in said nucleic acid linker portion.

46. The construct according to any of items 41 to 45, which comprises a phosphorothioate or phosphorodithioate internucleotide linkage linking:

the first nucleic acid portion of (a) to the nucleic acid linker portion as defined in items 16 (iii); and/or the second nucleic acid portion of (b) to the nucleic acid linker portion as defined in items 16 (iii); and/or the third nucleic acid portion of (c) to the nucleic acid linker portion as defined in items 16 (iii) and/or the fourth nucleic acid portion of (d) to the nucleic acid linker portion as defined in items 16 (iii); and/or the 1 to 8 additional nucleic acid portions as defined in items 14 or 15 to the nucleic acid linker portion as defined in items 16 (iii); and/or the passenger nucleic acid portions as defined in items 14 or 15 to the nucleic acid linker portion as defined in items 16 (iii).

47. The construct according to any of items 1 to 46, wherein at least one nucleotide of at least one of the following is modified:

the first nucleic acid portion of (a); and/or the second nucleic acid portion of (b); and/or the third nucleic acid portion of (c); and/or the fourth nucleic acid portion of (d); and/or to the extent present, the 1 to 8 additional nucleic acid portions as defined in items 14 or 15; and/or to the extent present, the passenger nucleic acid portions as defined in items 14 or 15; and/or to the extent present, the nucleic acid linker portion as defined in items 16 (iii).

48. The construct according to item 47, wherein one or more of the odd numbered nucleotides starting from the 5′ region of one of the following are modified, and/or wherein one or more of the even numbered nucleotides starting from the 5′ region of one of the following are modified, wherein typically the modification of the even numbered nucleotides is a second modification that is different from the modification of odd numbered nucleotides: the first nucleic acid portion of (a); and/or the second nucleic acid portion of (b); and/or the third nucleic acid portion of (c); and/or the fourth nucleic acid portion of (d); and/or to the extent present, the 1 to 8 additional nucleic acid portions as defined in items 14 or 15; and/or to the extent present, the passenger nucleic acid portions as defined in items 14 or 15.

49. The construct according to item 47 or 48, wherein one or more of the odd numbered nucleotides starting from the 3′ region of the third nucleic acid portion of (c) are modified by a modification that is different from the modification of odd numbered nucleotides starting from the 5′ region of the first nucleic acid portion of (a); and/or wherein one or more of the odd numbered nucleotides starting from the 3′ region of the fourth nucleic acid portion of (d) are modified by a modification that is different from the modification of odd numbered nucleotides starting from the 5′ region of the second nucleic acid portion of (b); and/or wherein one or more of the odd numbered nucleotides starting from the 3′ region of the passenger nucleic acid portions as defined in items 14 or 15, to the extent present, are modified by a modification that is different from the modification of odd numbered nucleotides starting from the 5′ region of the 1 to 8 additional nucleic acid portions as defined in items 14 or 15; and/or wherein one or more of the nucleotides of a nucleic acid linker portion as defined in items 16 (iii), to the extent present, are modified by a modification that (i) is different from the modification of an adjacent nucleotide of the 3′ region of the first nucleic acid portion of (a); and/or (ii) is different from the modification of an adjacent nucleotide of the 3′ region of the second nucleic acid portion of (b); and/or is different from the modification of an adjacent nucleotide of the 3′ region of the 1 to 8 additional nucleic acid portions, to the extent present, as defined in items 14 or 15.

50. The construct according to any of items 47 to 49, wherein one or more of the even numbered nucleotides starting from the 3′ region of: (i) the third nucleic acid portion of (c), and/or (ii) the fourth nucleic acid portion of (d), and/or (iii) said passenger nucleic acid portions as defined in items 14 or 15, to the extent present, are modified by a modification that is different from the modification of odd numbered nucleotides starting from the 3′ region of these respective portions.

51. The construct according to any of items 47 to 50, wherein at least one or more of the modified even numbered nucleotides of (i) the first nucleic acid portion of (a), and/or (ii) the second nucleic acid portion of (b), and/or (iii), to the extent present, the 1 to 8 additional nucleic acid portions as defined in items 14 or 15, is adjacent to at least one or more differently modified odd numbered nucleotides of these respective portions.

52. The construct according to any of items 47 to 51, wherein at least one or more of the modified even numbered nucleotides of (i) the third nucleic acid portion of (c), and/or (ii) the fourth nucleic acid portion of (d), and/or (iii), to the extent present, the passenger nucleic acid portions as defined in items 14 or 15, is adjacent to at least one or more differently modified odd numbered nucleotides of these respective portions.

53. The construct according to any of items 47 to 52, wherein a plurality of adjacent nucleotides of (i) the first nucleic acid portion of (a), and/or (ii) the second nucleic acid portion of (b), and/or (iii), to the extent present, the 1 to 8 additional nucleic acid portions as defined in claim 14 or 15, are modified by a common modification.

54. The construct according to any of items 47 to 53, wherein a plurality of adjacent nucleotides of (i) the third nucleic acid portion of (c), and/or (ii) the fourth nucleic acid portion of (d), and/or (iii), to the extent present, the passenger nucleic acid portions as defined in claim 14 or 15, are modified by a common modification.

55. The construct according to item 53 or 54, wherein said plurality of adjacent commonly modified nucleotides are 2 to 4 adjacent nucleotides, optionally 3 or 4 adjacent nucleotides.

56. The construct according to item 55, wherein said plurality of adjacent commonly modified nucleotides are located in the 5′ region of (i) the third nucleic acid portion of (c), and/or (ii) the fourth nucleic acid portion of (d), and/or (iii), to the extent present, the passenger nucleic acid portions as defined in items 14 or 15.

57. The construct according to any one of items 53 to 56, wherein a plurality of adjacent commonly modified nucleotides are located in the nucleic acid linker portion as defined in item 16 (iii).

58. The construct according to any of items 47 to 57, wherein the one or more of the modified nucleotides of first nucleic acid portion of (a) do not have a common modification present in the corresponding nucleotide of the third nucleic acid portion of (c) of the first duplex region; and/or one or more of the modified nucleotides of second nucleic acid portion of (b) do not have a common modification present in the corresponding nucleotide of the fourth nucleic acid portion of (d) of the second duplex region; and/or one or more of the modified nucleotides of the 1 to 8 additional nucleic acid portions, to the extent present, as defined in item 14 or 15, do not have a common modification present in the corresponding nucleotide of the corresponding passenger nucleic acid portions of the respective duplex regions.

59. The construct according to any of items 47 to 58, wherein the one or more of the modified nucleotides of the first nucleic acid portion of (a) are shifted by at least one nucleotide relative to a commonly modified nucleotide of the third nucleic acid portion of (c); and/or one or more of the modified nucleotides of the second nucleic acid portion of (b) are shifted by at least one nucleotide relative to a commonly modified nucleotide of the fourth nucleic acid portion of (d); and/or one or more of the modified nucleotides of the 1 to 8 additional nucleic acid portions, to the extent present, as defined in item 14 or 15 are shifted by at least one nucleotide relative to a commonly modified nucleotide of the passenger nucleic acid portions, to the extent present, as defined in item 14 or 15.

60. The construct according to any of items 47 to 59, wherein the modification and/or modifications are each and individually sugar, phosphate, or base modifications.

61. The construct according to item 60, where the modification is selected from nucleotides with 2′ modified sugars; conformationally restricted nucleotides (CRN) sugar such as locked nucleic acid (LNA), (S)-constrained ethyl bicyclic nucleic acid, and constrained ethyl (cEt), tricyclo-DNA; morpholino, unlocked nucleic acid (UNA), glycol nucleic acid (GNA), D-hexitol nucleic acid (HNA), and cyclohexene nucleic acid (CeNA).

62. The construct according to item 61, wherein said 2′ modified sugar is selected from 2′-O-alkyl modified sugar, 2′-O-methyl modified sugar, 2′-O-methoxyethyl modified sugar, 2′-O-allyl modified sugar, 2′-C-allyl modified sugar, 2′-deoxy modified sugar such as 2′-deoxy ribose, 2′-F modified sugar, 2′-arabino-fluoro modified sugar, 2′-O-benzyl modified sugar, 2′-amino modified sugar, and 2′-O-methyl-4-pyridine modified sugar.

63. The construct according to any of items 60 to 62, wherein the base modification is any one of an abasic nucleotide and a non-natural base comprising nucleotide.

64. The construct according to any of items 47 to 63, wherein at least one modification is a 2′-O-methyl modification in a ribose moiety.

65. The construct according to any of items 47 to 64, wherein at least one modification is a 2′-F modification in a ribose moiety.

66. The construct according to any of items 47 to 65 wherein the nucleotides at any of positions 2 and 14 downstream from the first nucleotide of the 5′ region of (i) the first nucleic acid portion of (a); and/or (ii) the second nucleic acid portion of (b); and/or (iii), to the extent present, the 1 to 8 additional nucleic acid portions as defined in item 14 or 15; do not contain 2′-O-methyl modifications in ribose moieties.

67. The construct according to any of items 47 to 66, wherein one, two or all three nucleotides of (i) the third nucleic acid portion of (c); and/or (ii) the fourth nucleic acid portion of (d); and/or (iii), to the extent present, said passenger nucleic acid portions as defined in item 14 or 15; that respectively correspond in position to any of the nucleotides at any of positions 11 to 13 downstream from the first nucleotide of the 5′ region of (i) the first nucleic acid portion of (a); and/or (ii) the second nucleic acid portion of (b); and/or (iii) the 1 to 8 additional nucleic acid portions, to the extent present, as defined in item 14 or 15; do not contain 2′-O-methyl modifications in ribose moieties.

68. The construct according to item 66 or 67, wherein the nucleotides at any of positions 2 and 14 downstream from the first of (i) the first nucleic acid portion of (a); and/or (ii) the second nucleic acid portion of (b); and/or (iii), to the extent present, the 1 to 8 additional nucleic acid portions as defined in item 14 or 15; contain 2′-F modifications in ribose moieties.

69. The construct according to any of items 66 to 68, wherein one, two or all three nucleotides of (i) the third nucleic acid portion of (c); and or (ii) the fourth nucleic acid portion of (d); and/or (iii), to the extent present, said passenger nucleic acid portions as defined in items 14 or 15; that respectively correspond in position to any of the nucleotides at any of positions 11 to 13 downstream from the first nucleotide of the 5′ region of (i) the first nucleic acid portion of (a); and/or (ii) the second nucleic acid portion of (b); and/or (iii), to the extent present, the 1 to 8 additional nucleic acid portions as defined in item 14 or 15; contain 2′-F modifications in ribose moieties.

70. The construct according to any one of items 65 to 69, wherein all remaining nucleotides contain either 2′-O-methyl modifications or 2′-F modifications in ribose moieties, optionally with the exception of the unmodified nucleotide(s) in accordance with item 5.

71. The construct according to item 70, wherein said remaining nucleotides contain 2′-O-methyl modifications in ribose moieties.

72. The construct according to item 70 or 71, wherein said one or more, optionally one, unmodified nucleotide represents any of the nucleotides of the nucleic acid linker portion as defined in item 16 (iii), optionally the nucleotide of the nucleic acid linker portion as defined in item 16 (iii) that is adjacent to (i) the third nucleic acid portion of (c); and or (ii) the fourth nucleic acid portion of (d); and/or (iii), to the extent present, said passenger nucleic acid portions as defined in item 14 or 15.

73. The construct of any one of the preceding items, wherein

(a) said first nucleic acid portion is selected from Table 3a;

(b) said second nucleic acid portion is selected from Table 3b;

(c) said third nucleic acid portion is selected from Table 4a; and/or

(d) said fourth nucleic acid portion is selected from Table 4b.

74. The construct of any one of the preceding items wherein said construct comprises two strands, wherein the first strand is selected from Table 5a, such as table entry number 50, and the second strand from Table 5b, such as table entry number 50; or said first and second strands are selected from Table 7a, such as entries 1 (first strand) and 2 (second strand) or entries 3 (first strand) and 4 (second strand), respectively; or said first and second strands are selected from Table 7b, such as entries 11 (first strand) and 12 (second strand), or entries 13 (first strand) and 14 (second strand), or entries 19 (first strand) and 20 (second strand), respectively.

75. The construct of item 74, wherein first and second strands are selected from entries 2, 18, 5, 20, 50 to 54, 25, 33, and 45 of Tables 5a and 5b, respectively.

76. The construct according to any one of items 73 to 75, wherein the 3′ terminal positions of said first and said third nucleic acid portions are replaced with an unmodified nucleotide.

77. The construct according to any of items 1 to 76, which comprises at least one vinylphosphonate modification, such as at least one vinylphosphonate modification in the 5′ region of (i) the first nucleic acid portion of (a); and/or (ii) the second nucleic acid portion of (b); and/or (iii), to the extent present, the 1 to 8 additional nucleic acid portions as defined in item 14 o 15.

78. The construct according to any of items 1 to 77, wherein one or more nucleotides of the first nucleic acid portion of (a); and/or the second nucleic acid portion of (b); and/or the third nucleic acid portion of (c); and/or the fourth nucleic acid portion of (d); and/or to the extent present, the 1 to 8 additional nucleic acid portions as defined in item 14 or 15; and/or to the extent present, the passenger nucleic acid portions as defined in item 14 or 15; is an inverted nucleotide and is attached to the adjacent nucleotide via the 3′ carbon of the nucleotide and the 3′ carbon of the adjacent nucleotide, and/or is an inverted nucleotide and is attached to the adjacent nucleotide via the 5′ carbon of the nucleotide and the 5′ carbon of the adjacent nucleotide.

79. The construct according to item 78, wherein the inverted nucleotide is attached to the adjacent nucleotide via a phosphate group by way of a phosphodiester linkage; or is attached to the adjacent nucleotide via a phosphorothioate group; or is attached to the adjacent nucleotide via a phosphorodithioate group.

80. The construct according to any of items 1 to 79, which is blunt ended.

81. The construct according to any of items 1 to 79, wherein the first nucleic acid portion of (a); and/or the second nucleic acid portion of (b); and/or the third nucleic acid portion of (c); and/or the fourth nucleic acid portion of (d); and/or to the extent present, the 1 to 8 additional nucleic acid portions as defined in item 14 or 15; and/or to the extent present, the passenger nucleic acid portions as defined in item 14 or 15; has an overhang.

82. The construct according to any of items 1 to 81, wherein the target RNA is an mRNA or an other RNA molecule.

83. A composition comprising a construct according to any of items 1 to 82, and a physiologically acceptable excipient.

84. A pharmaceutical composition comprising a construct according to any of items 1 to 82.

85. The pharmaceutical composition of item 84, further comprising a pharmaceutically acceptable excipient, diluent, antioxidant, and/or preservative.

86. The pharmaceutical composition of item 84 or 85, wherein said construct is the only pharmaceutically active agent.

87. The pharmaceutical composition of item 86, wherein said pharmaceutical composition is to be administered to patients or individuals which are statin-intolerant and/or for whom statins are contraindicated.

88. The pharmaceutical composition of item 84 or 85, wherein said pharmaceutical composition furthermore comprises one or more further pharmaceutically active agents.

89. The pharmaceutical composition of item 88, wherein said further pharmaceutically active agent(s) is/are an RNAi agent which is directed to a target different from PCSK9 and from APOC3 and/or a lipid-lowering agent distinct from said construct, wherein said lipid-lowering agent is optionally ezetimib; Vascepa; Vupanorsen; statins such as Rosuvastatin and Simvastatin; and/or fibrates such fenofibrate.

90. The pharmaceutical composition of item 88 or 89, wherein said construct and said further pharmaceutically active agent(s) are to be administered concomitantly or in any order.

91. A construct according to any of items 1 to 82, for use in human or veterinary medicine or therapy.

92. A construct according to any of items 1 to 82, for use in a method of treating, ameliorating and/or preventing a disease or disorder.

93. The construct for use of item 92, wherein said disease or disorder is a PCSK9- and/or an APOC3-associated disease or disorder or a disease or disorder requiring reduction of PCSK9 and/or APOC3 expression levels.

94. The compound for use of item 92 or 93, wherein said disease or disorder is

(a) a PCSK9-associated disease or disorder, or a disease or disorder requiring reduction of low-density lipoprotein (LDL) cholesterol, said disease or disorder optionally being selected from dyslipidemia including mixed dyslipidemia, hypercholesterolemia, heterozygous familial hypercholesterolemia, non-familial hypercholesterolemia; atherosclerosis; and atherosclerotic cardiovascular disease (ASCVD) including myocardial infarction, stroke and peripheral arterial disease; and/or

(b) an APOC3-associated disease or disorder, or a disease or disorder requiring reduction of APOC3 expression levels, said disease or disorder optionally being selected from dyslipidemia including mixed dyslipidemia; hyperchylomicronemia including familial hyperchylomicronemia; hypertriglyceridemia, optionally severe hypertriglyceridemia and/or hypertriglyceridemia with blood triglyceride levels above 500 mg/dl; inflammation including low-grade inflammation; atherosclerosis; atherosclerotic cardiovascular diseases (ASCVD) including major adverse cardiovascular events (MACE) such as myocardial infarction, stroke and peripheral arterial disease; and pancreatitis including acute pancreatitis.

95. A method of treating a disease or disorder comprising administration of a construct according to any of items 1 to 82, to an individual in need of treatment.

96. The method according to item 95, wherein the construct is administered subcutaneously or intravenously to the individual, optionally subcutaneously.

97. The method according to item 95 or 96, wherein subsequent to in vivo administration the construct disassembles to yield at least first and second discrete nucleic acid targeting molecules that target portions of RNA transcribed from a PCSK9 and an APOC3 gene, respectively.

98. A process of making a construct according to any of items 1 to 82.

99. A process according to item 98, which comprises:

(i) synthesizing each of:

(a) a first nucleic acid portion that is at least partially complementary to at least a first portion of RNA transcribed from a target gene;

(b) a second nucleic acid portion that is at least partially complementary to at least a second portion of RNA transcribed from a target gene, which target gene may be the same or different to the target gene defined in (a);

(c) a third nucleic acid portion that is at least partially complementary to said first nucleic acid portion of (a);

(d) a fourth nucleic acid portion that is at least partially complementary to said second nucleic acid portion of (b);

(ii) contacting at least said first and second nucleic acid portions of (a) and (b) in vitro, so as to form a first nucleic acid duplex region comprising said first and second nucleic acid portions of (a) and (b);

(iii) contacting at least said third and fourth nucleic acid portions of (c) and (d) in vitro, so as to form a second nucleic acid duplex region comprising said third and fourth nucleic acid portions of (c) and (d);

(iv) forming a nucleic acid construct in vitro comprising at least said first and second nucleic acid duplex regions.

100. A process according to item 99, which further comprises generating from said construct at least first and second nucleic acid targeting molecules, wherein the first nucleic acid targeting molecule modulates expression of the target gene of (a), and comprises, or is derived from, at least the first nucleic acid portion of (a), and wherein the second nucleic acid targeting molecule modulates expression of said target gene of (b), and comprises, or is derived from, the second nucleic acid portion of (b).

101. A process according to item 100, wherein said at least first and second nucleic acid targeting molecules are generated subsequent to in vivo administration.

102. A process according to item 101, wherein labile functionality present in said construct is cleaved subsequent to in vivo administration so as to generate said at least first and second discrete nucleic acid targeting molecules.

103. A process according to item 102, wherein said labile functionality comprises one or more unmodified nucleotides.

104. A process according to item 103, wherein said one or more unmodified nucleotides of said labile functionality represent one or more cleavage positions within said construct whereby subsequent to in vivo administration said construct is cleaved at said one or more cleavage positions so as to yield said at least first and second discrete nucleic acid targeting molecules.

105. A process according to item 104, wherein said cleavage positions are respectively located within the construct so that subsequent to cleavage said first discrete nucleic acid targeting molecule comprises, or is derived from, said first nucleic acid duplex region, and said second discrete nucleic acid targeting molecule comprises, or is derived from, said second nucleic acid duplex region.

The following Tables show nucleobase sequences and full definitions (including sugar modifications and, where applicable, phosphate modifications) of portions as well as of entire constructs as described herein.

The notation used is common in the art and as the following meaning:

A represents adenine;

U represents uracil;

C represents cytosine;

G represents guanine.

P represents a terminal phosphate group which may or may not be present;

m represents a methyl modification at the 2′ position of the sugar of the underlying nucleoside, wherein an accordingly modified nucleotide such as mG is sometimes displayed in brackets ([mG]);

f represents a fluoro modification at the 2′ position of the sugar of the underlying nucleoside, wherein an accordingly modified nucleotide such as fG is sometimes displayed in brackets ([fG]);

r indicates an unmodified (2′-OH) ribonucleotide, wherein corresponding nucleotide such as rG is sometimes displayed in brackets ([rG]);

(ps), #, [#], or * represents a phosphorothioate inter-nucleoside linkage;

i represents an inverted inter-nucleoside linkage, which can be either 3′-3′, or 5′-5′;

vp represents vinyl phosphonate;

mvp represents methyl vinyl phosphonate;

3×GalNAc represents an optionally present trivalent GalNAc; and

Mono-GalNAc-PA, represents an optional one of optionally three GalNAc bearing moieties, the assembly of three Mono-GalNAc-PA moieties also being referred to as “toothbrush”, where the individual moieties are connected by phosphoramidates (“PA”); see the embodiments for an illustration.

Table 1a below shows the nucleobase sequences of PCSK9-targeting antisense portions (first nucleic acid portions). The sequences are those of SEQ ID NOs: 1 to 7 (same order).

| Sequence | Anti-Sense Sequence |
|---|---|
| ID | (5′ to 3′) |
| PCS44 | UCUUCAAGUUACAAAAGCA |
| PCS46 | UAAAAGCAAAACAGGUCUA |
| PCS53 | UACAAAAGCAAAACAGGUC |
| PCS29 | UGCAAAACAGGUCUAGAAA |
| PCS57 | UCAAAAGCAAAACAGGUCU |
| PCS52 | UAGAAUAAAUAUCUUCAAG |
| PCS55 | UAAAGCAAAACAGGUCUAG |

Table 1b below shows the nucleobase sequences of APOC3-targeting antisense portions (second nucleic acid portions). The sequences are those of SEQ ID NOs: 8 to 14 (same order). The nucleobase sequence of a further APOC3-targeting antisense portion of the molecules is set forth in SEQ ID NO: 29.

| Sequence | Anti-Sense Sequence |
|---|---|
| ID | (5′ to 3′) |
| AP277 | UUGGAUAGGCAGGUGGACU |
| AP337 | UGCACUGAGAAUACUGUCC |
| AP028 | UCAACAAGGAGUACCCGGG |
| AP369 | UCUUGUCCAGCUUUAUUGG |
| AP366 | UUCCAGCUUUAUUGGGAGG |
| AP367 | UGUCCAGCUUUAUUGGGAG |
| AP336 | UCACUGAGAAUACUGUCCC |

Table 2a below shows the nucleobase sequences of PCSK9-targeting sense portions (third nucleic acid portions). The sequences are those of SEQ ID NOs:15 to 21 (same order).

| Sequence | |
|---|---|
| ID | SS Sequence (5′ to 3′) |
| PCS44 | UUUGUAACUUGAAGA |
| PCS46 | CCUGUUUUGCUUUUA |
| PCS53 | UGUUUUGCUUUUGUA |
| PCS29 | UAGACCUGUUUUGCA |
| PCS57 | CUGUUUUGCUUUUGA |
| PCS52 | AAGAUAUUUAUUCUA |
| PCS55 | ACCUGUUUUGCUUUA |

Table 2b below shows the nucleobase sequences of APOC3-targeting sense portions (fourth nucleic acid portions). The sequences are those of SEQ ID NOs: 22 to 28 (same order). The nucleobase sequence of a further APOC3-targeting sense portion of the molecule is set forth in SEQ ID NO: 30.

| Sequence | |
|---|---|
| ID | SS Sequence (5′ to 3′) |
| AP277 | CACCUGCCUAUCCAA |
| AP337 | AGUAUUCUCAGUGCA |
| AP028 | GGUACUCCUUGUUGA |
| AP369 | UAAAGCUGGACAAGA |
| AP366 | CCAAUAAAGCUGGAA |
| AP367 | CAAUAAAGCUGGACA |
| AP336 | CAGUAUUCUCAGUGA |

Table 3a shows PCSK9-targeting antisense portions including sugar modification information.

| SEQ ID NO: | AS Modified |
|---|---|
| 33 | PmU.fC.mU.fU.mC.fA.mA.fG.mU.fU.mA.fC.mA.fA.mA.fA.mG.fC.mA |
| 34 | PmU.fA.mA.fA.mA.fG.mC.fA.mA.fA.mA.fC.mA.fG.mG.fU.mC.fU.mA |
| 35 | PmU.fA.mC.fA.mA.fA.mA.fG.mC.fA.mA.fA.mA.fC.mA.fG.mG.fU.mC |
| 36 | PmU.fG.mC.fA.mA.fA.mA.fC.mA.fG.mG.fU.mC.fU.mA.fG.mA.fA.mA |
| 37 | PmU.fC.mA.fA.mA.fA.mG.fC.mA.fA.mA.fA.mC.fA.mG.fG.mU.fC.mU |
| 38 | PmU.fA.mG.fA.mA.fU.mA.fA.mA.fU.mA.fU.mC.fU.mU.fC.mA.fA.mG |
| 39 | PmU.fA.mA.fA.mG.fC.mA.fA.mA.fA.mC.fA.mG.fG.mU.fC.mU.fA.mG |

Table 3b shows APOC3-targeting antisense portions including sugar modification information.

| SEQ ID NO: | AS Modified |
|---|---|
| 40 | PmU.fU.mG.fG.mA.fU.mA.fG.mG.fC.mA.fG.mG.fU.mG.fG.mA.fC.mU |
| 41 | PmU.fG.mC.fA.mC.fU.mG.fA.mG.fA.mA.fU.mA.fC.mU.fG.mU.fC.mC |
| 42 | PmU.fC.mA.fA.mC.fA.mA.fG.mG.fA.mG.fU.mA.fC.mC.fC.mG.fG.mG |
| 43 | PmU.fC.mU.fU.mG.fU.mC.fC.mA.fG.mC.fU.mU.fU.mA.fU.mU.fG.mG |
| 44 | PmU.fU.mC.fC.mA.fG.mC.fU.mU.fU.mA.fU.mU.fG.mG.fG.mA.fG.mG |
| 45 | PmU.fG.mU.fC.mC.fA.mG.fC.mU.fU.mU.fA.mU.fU.mG.fG.mG.fA.mG |
| 46 | PmU.fC.mA.fC.mU.fG.mA.fG.mA.fA.mU.fA.mC.fU.mG.fU.mC.fC.mC |

Table 4a shows PCSK9-targeting sense portions including sugar modification information.

| SEQ ID | |
|---|---|
| NO: | S Modified |
| 47 | fU.mU.fU.mG.fU.mA.fA.mC.fU.mU.fG.mA.fA.mG.fA |
| 48 | fC.mC.fU.mG.fU.mU.fU.mU.fG.mC.fU.mU.fU.mU.fA |
| 49 | fU.mG.fU.mU.fU.mU.fG.mC.fU.mU.fU.mU.fG.mU.fA |
| 50 | fU.mA.fG.mA.fC.mC.fU.mG.fU.mU.fU.mU.fG.mC.fA |
| 51 | fC.mU.fG.mU.fU.mU.fU.mG.fC.mU.fU.mU.fU.mG.fA |
| 52 | fA.mA.fG.mA.fU.mA.fU.mU.fU.mA.fU.mU.fC.mU.fA |
| 53 | fA.mC.fC.mU.fG.mU.fU.mU.fU.mG.fC.mU.fU.mU.fA |

Table 4b shows APOC3-targeting sense portions including sugar modification information.

| SEQ ID | |
|---|---|
| NO: | S Modified |
| 54 | fC.mA.fC.mC.fU.mG.fC.mC.fU.mA.fU.mC.fC.mA.fA |
| 55 | fA.mG.fU.mA.fU.mU.fC.mU.fC.mA.fG.mU.fG.mC.fA |
| 56 | fG.mG.fU.mA.fC.mU.fC.mC.fU.mU.fG.mU.fU.mG.fA |
| 57 | fU.mA.fA.mA.fG.mC.fU.mG.fG.mA.fC.mA.fA.mG.fA |
| 58 | fC.mC.fA.mA.fU.mA.fA.mA.fG.mC.fU.mG.fG.mA.fA |
| 59 | fC.mA.fA.mU.fA.mA.fA.mG.fC.mU.fG.mG.fA.mC.fA |
| 60 | fC.mA.fG.mU.fA.mU.fU.mC.fU.mC.fA.mG.fU.mG.fA |

Table 5a shows linked first and fourth nucleic acid portions of the molecules. Linking is direct to give rise to a single contiguous strand.

| | | | |
|---|---|---|---|
| SEQ ID | 1 | AP277AS/PCS44 | PmU.fC.mU.fU.mC.fA.mA.fG.mU.fU.mA.fC.mA.fA.mA.fA. |
| NO: 61 | | | mG.fC.mA.fC.mA.fC.mC.fU.mG.fC.mC.fU.mA.fU.mC.fC. |
| | | | mA.fA |
| SEQ ID | 2 | AP277AS/PCS46 | PmU.fA.mA.fA.mA.fG.mC.fA.mA.fA.mA.fC.mA.fG.mG.fU. |
| NO: 62 | | | mC.fU.mA.fC.mA.fC.mC.fU.mG.fC.mC.fU.mA.fU.mC.fC. |
| | | | mA.fA |
| SEQ ID | 3 | AP277AS/PCS53 | PmU.fA.mC.fA.mA.fA.mA.fG.mC.fA.mA.fA.mA.fC.mA.fG. |
| NO: 63 | | | mG.fU.mC.fC.mA.fC.mC.fU.mG.fC.mC.fU.mA.fU.mC.fC. |
| | | | mA.fA |
| SEQ ID | 4 | AP277AS/PCS29 | PmU.fG.mC.fA.mA.fA.mA.fC.mA.fG.mG.fU.mC.fU.mA.fG. |
| NO: 64 | | | mA.fA.mA.fC.mA.fC.mC.fU.mG.fC.mC.fU.mA.fU.mC.fC. |
| | | | mA.fA |
| SEQ ID | 5 | AP277AS/PCS57 | PmU.fC.mA.fA.mA.fA.mG.fC.mA.fA.mA.fA.mC.fA.mG.fG. |
| NO: 65 | | | mU.fC.mU.fC.mA.fC.mC.fU.mG.fC.mC.fU.mA.fU.mC.fC. |
| | | | mA.fA |
| SEQ ID | 6 | AP277AS/PCS52 | PmU.fA.mG.fA.mA.fU.mA.fA.mA.fU.mA.fU.mC.fU.mU.fC. |
| NO: 66 | | | mA.fA.mG.fC.mA.fC.mC.fU.mG.fC.mC.fU.mA.fU.mC.fC. |
| | | | mA.fA |
| SEQ ID | 7 | AP277AS/PCS55 | PmU.fA.mA.fA.mG.fC.mA.fA.mA.fA.mC.fA.mG.fG.mU.fC. |
| NO: 67 | | | mU.fA.mG.fC.mA.fC.mC.fU.mG.fC.mC.fU.mA.fU.mC.fC. |
| | | | mA.fA |
| SEQ ID | 8 | AP377As/PCS44 | PmU.fC.mU.fU.mC.fA.mA.fG.mU.fU.mA.fC.mA.fA.mA.fA. |
| NO: 68 | | | mG.fC.mA.fA.mG.fU.mA.fU.mU.fC.mU.fC.mA.fG.mU.fG. |
| | | | mC.fA |
| SEQ ID | 9 | AP377As/PCS46 | PmU.fA.mA.fA.mA.fG.mC.fA.mA.fA.mA.fC.mA.fG.mG.fU. |
| NO: 69 | | | mC.fU.mA.fA.mG.fU.mA.fU.mU.fC.mU.fC.mA.fG.mU.fG. |
| | | | mC.fA |
| SEQ ID | 10 | AP377As/PCS53 | PmU.fA.mC.fA.mA.fA.mA.fG.mC.fA.mA.fA.mA.fC.mA.fG. |
| NO: 70 | | | mG.fU.mC.fA.mG.fU.mA.fU.mU.fC.mU.fC.mA.fG.mU.fG. |
| | | | mC.fA |
| SEQ ID | 11 | AP377As/PCS29 | PmU.fG.mC.fA.mA.fA.mA.fC.mA.fG.mG.fU.mC.fU.mA.fG. |
| NO: 71 | | | mA.fA.mA.fA.mG.fU.mA.fU.mU.fC.mU.fC.mA.fG.mU.fG. |
| | | | mC.fA |
| SEQ ID | 12 | AP377As/PCS57 | PmU.fC.mA.fA.mA.fA.mG.fC.mA.fA.mA.fA.mC.fA.mG.fG. |
| NO: 72 | | | mU.fC.mU.fA.mG.fU.mA.fU.mU.fC.mU.fC.mA.fG.mU.fG. |
| | | | mC.fA |
| SEQ ID | 13 | AP377As/PCS52 | PmU.fA.mG.fA.mA.fU.mA.fA.mA.fU.mA.fU.mC.fU.mU.fC. |
| NO: 73 | | | mA.fA.mG.fA.mG.fU.mA.fU.mU.fC.mU.fC.mA.fG.mU.fG. |
| | | | mC.fA |
| SEQ ID | 14 | AP377As/PCS55 | PmU.fA.mA.fA.mG.fC.mA.fA.mA.fA.mC.fA.mG.fG.mU.fC. |
| NO: 74 | | | mU.fA.mG.fA.mG.fU.mA.fU.mU.fC.mU.fC.mA.fG.mU.fG. |
| | | | mC.fA |
| SEQ ID | 15 | AP028As/PCS44 | PmU.fC.mU.fU.mC.fA.mA.fG.mU.fU.mA.fC.mA.fA.mA.fA. |
| NO: 75 | | | mG.fC.mA.fG.mG.fU.mA.fC.mU.fC.mC.fU.mU.fG.mU.fU. |
| | | | mG.fA |
| SEQ ID | 16 | AP028As/PCS46 | PmU.fA.mA.fA.mA.fG.mC.fA.mA.fA.mA.fC.mA.fG.mG.fU. |
| NO: 76 | | | mC.fU.mA.fG.mG.fU.mA.fC.mU.fC.mC.fU.mU.fG.mU.fU. |
| | | | mG.fA |
| SEQ ID | 17 | AP028As/PCS53 | PmU.fA.mC.fA.mA.fA.mA.fG.mC.fA.mA.fA.mA.fC.mA.fG. |
| NO: 77 | | | mG.fU.mC.fG.mG.fU.mA.fC.mU.fC.mC.fU.mU.fG.mU.fU. |
| | | | mG.fA |
| SEQ ID | 18 | AP028As/PCS29 | PmU.fG.mC.fA.mA.fA.mA.fC.mA.fG.mG.fU.mC.fU.mA.fG. |
| NO: 78 | | | mA.fA.mA.fG.mG.fU.mA.fC.mU.fC.mC.fU.mU.fG.mU.fU. |
| | | | mG.fA |
| SEQ ID | 19 | AP028As/PCS57 | PmU.fC.mA.fA.mA.fA.mG.fC.mA.fA.mA.fA.mC.fA.mG.fG. |
| NO: 79 | | | mU.fC.mU.fG.mG.fU.mA.fC.mU.fC.mC.fU.mU.fG.mU.fU. |
| | | | mG.fA |
| SEQ ID | 20 | AP028As/PCS52 | PmU.fA.mG.fA.mA.fU.mA.fA.mA.fU.mA.fU.mC.fU.mU.fC. |
| NO: 80 | | | mA.fA.mG.fG.mG.fU.mA.fC.mU.fC.mC.fU.mU.fG.mU.fU. |
| | | | mG.fA |
| SEQ ID | 21 | AP028As/PCS55 | PmU.fA.mA.fA.mG.fC.mA.fA.mA.fA.mC.fA.mG.fG.mU.fC. |
| NO: 81 | | | mU.fA.mG.fG.mG.fU.mA.fC.mU.fC.mC.fU.mU.fG.mU.fU. |
| | | | mG.fA |
| SEQ ID | 22 | AP369As/PCS44 | PmU.fC.mU.fU.mC.fA.mA.fG.mU.fU.mA.fC.mA.fA.mA.fA. |
| NO: 82 | | | mG.fC.mA.fU.mA.fA.mA.fG.mC.fU.mG.fG.mA.fC.mA.fA. |
| | | | mG.fA |
| SEQ ID | 23 | AP369As/PCS46 | PmU.fA.mA.fA.mA.fG.mC.fA.mA.fA.mA.fC.mA.fG.mG.fU. |
| NO: 83 | | | mC.fU.mA.fU.mA.fA.mA.fG.mC.fU.mG.fG.mA.fC.mA.fA. |
| | | | mG.fA |
| SEQ ID | 24 | AP369As/PCS53 | PmU.fA.mC.fA.mA.fA.mA.fG.mC.fA.mA.fA.mA.fC.mA.fG. |
| NO: 84 | | | mG.fU.mC.fU.mA.fA.mA.fG.mC.fU.mG.fG.mA.fC.mA.fA. |
| | | | mG.fA |
| SEQ ID | 25 | AP369As/PCS29 | PmU.fG.mC.fA.mA.fA.mA.fC.mA.fG.mG.fU.mC.fU.mA.fG. |
| NO: 85 | | | mA.fA.mA.fU.mA.fA.mA.fG.mC.fU.mG.fG.mA.fC.mA.fA. |
| | | | mG.fA |
| SEQ ID | 26 | AP369As/PCS57 | PmU.fC.mA.fA.mA.fA.mG.fC.mA.fA.mA.fA.mC.fA.mG.fG. |
| NO: 86 | | | mU.fC.mU.fU.mA.fA.mA.fG.mC.fU.mG.fG.mA.fC.mA.fA. |
| | | | mG.fA |
| SEQ ID | 27 | AP369As/PCS52 | PmU.fA.mG.fA.mA.fU.mA.fA.mA.fU.mA.fU.mC.fU.mU.fC. |
| NO: 87 | | | mA.fA.mG.fU.mA.fA.mA.fG.mC.fU.mG.fG.mA.fC.mA.fA. |
| | | | mG.fA |
| SEQ ID | 28 | AP369As/PCS55 | PmU.fA.mA.fA.mG.fC.mA.fA.mA.fA.mC.fA.mG.fG.mU.fC. |
| NO: 88 | | | mU.fA.mG.fU.mA.fA.mA.fG.mC.fU.mG.fG.mA.fC.mA.fA. |
| | | | mG.fA |
| SEQ ID | 29 | AP366AS/PCS44 | PmU.fC.mU.fU.mC.fA.mA.fG.mU.fU.mA.fC.mA.fA.mA.fA. |
| NO: 89 | | | mG.fC.mA.fC.mC.fA.mA.fU.mA.fA.mA.fG.mC.fU.mG.fG. |
| | | | mA.fA |
| SEQ ID | 30 | AP366AS/PCS46 | PmU.fA.mA.fA.mA.fG.mC.fA.mA.fA.mA.fC.mA.fG.mG.fU. |
| NO: 90 | | | mC.fU.mA.fC.mC.fA.mA.fU.mA.fA.mA.fG.mC.fU.mG.fG. |
| | | | mA.fA |
| SEQ ID | 31 | AP366AS/PCS53 | PmU.fA.mC.fA.mA.fA.mA.fG.mC.fA.mA.fA.mA.fC.mA.fG. |
| NO: 91 | | | mG.fU.mC.fC.mC.fA.mA.fU.mA.fA.mA.fG.mC.fU.mG.fG. |
| | | | mA.fA |
| SEQ ID | 32 | AP366AS/PCS29 | PmU.fG.mC.fA.mA.fA.mA.fC.mA.fG.mG.fU.mC.fU.mA.fG. |
| NO: 92 | | | mA.fA.mA.fC.mC.fA.mA.fU.mA.fA.mA.fG.mC.fU.mG.fG. |
| | | | mA.fA |
| SEQ ID | 33 | AP366AS/PCS57 | PmU.fC.mA.fA.mA.fA.mG.fC.mA.fA.mA.fA.mC.fA.mG.fG. |
| NO: 93 | | | mU.fC.mU.fC.mC.fA.mA.fU.mA.fA.mA.fG.mC.fU.mG.fG. |
| | | | mA.fA |
| SEQ ID | 34 | AP366AS/PCS52 | PmU.fA.mG.fA.mA.fU.mA.fA.mA.fU.mA.fU.mC.fU.mU.fC. |
| NO: 94 | | | mA.fA.mG.fC.mC.fA.mA.fU.mA.fA.mA.fG.mC.fU.mG.fG. |
| | | | mA.fA |
| SEQ ID | 35 | AP366AS/PCS55 | PmU.fA.mA.fA.mG.fC.mA.fA.mA.fA.mC.fA.mG.fG.mU.fC. |
| NO: 95 | | | mU.fA.mG.fC.mC.fA.mA.fU.mA.fA.mA.fG.mC.fU.mG.fG. |
| | | | mA.fA |
| SEQ ID | 36 | AP367As/PCS44 | PmU.fC.mU.fU.mC.fA.mA.fG.mU.fU.mA.fC.mA.fA.mA.fA. |
| NO: 96 | | | mG.fC.mA.fC.mA.fA.mU.fA.mA.fA.mG.fC.mU.fG.mG.fA. |
| | | | mC.fA |
| SEQ ID | 37 | AP367As/PCS46 | PmU.fA.mA.fA.mA.fG.mC.fA.mA.fA.mA.fC.mA.fG.mG.fU. |
| NO: 97 | | | mC.fU.mA.fC.mA.fA.mU.fA.mA.fA.mG.fC.mU.fG.mG.fA. |
| | | | mC.fA |
| SEQ ID | 38 | AP367As/PCS53 | PmU.fA.mC.fA.mA.fA.mA.fG.mC.fA.mA.fA.mA.fC.mA.fG. |
| NO: 98 | | | mG.fU.mC.fC.mA.fA.mU.fA.mA.fA.mG.fC.mU.fG.mG.fA. |
| | | | mC.fA |
| SEQ ID | 39 | AP367As/PCS29 | PmU.fG.mC.fA.mA.fA.mA.fC.mA.fG.mG.fU.mC.fU.mA.fG. |
| NO: 99 | | | mA.fA.mA.fC.mA.fA.mU.fA.mA.fA.mG.fC.mU.fG.mG.fA. |
| | | | mC.fA |
| SEQ ID | 40 | AP367As/PCS57 | PmU.fC.mA.fA.mA.fA.mG.fC.mA.fA.mA.fA.mC.fA.mG.fG. |
| NO: 100 | | | mU.fC.mU.fC.mA.fA.mU.fA.mA.fA.mG.fC.mU.fG.mG.fA. |
| | | | mC.fA |
| SEQ ID | 41 | AP367As/PCS52 | PmU.fA.mG.fA.mA.fU.mA.fA.mA.fU.mA.fU.mC.fU.mU.fC. |
| NO: 101 | | | mA.fA.mG.fC.mA.fA.mU.fA.mA.fA.mG.fC.mU.fG.mG.fA. |
| | | | mC.fA |
| SEQ ID | 42 | AP367As/PCS55 | PmU.fA.mA.fA.mG.fC.mA.fA.mA.fA.mC.fA.mG.fG.mU.fC. |
| NO: 102 | | | mU.fA.mG.fC.mA.fA.mU.fA.mA.fA.mG.fC.mU.fG.mG.fA. |
| | | | mC.fA |
| SEQ ID | 43 | AP336AS/PCS44 | PmU.fC.mU.fU.mC.fA.mA.fG.mU.fU.mA.fC.mA.fA.mA.fA. |
| NO: 103 | | | mG.fC.mA.fC.mA.fG.mU.fA.mU.fU.mC.fU.mC.fA.mG.fU. |
| | | | mG.fA |
| SEQ ID | 44 | AP336AS/PCS46 | PmU.fA.mA.fA.mA.fG.mC.fA.mA.fA.mA.fC.mA.fG.mG.fU. |
| NO: 104 | | | mC.fU.mA.fC.mA.fG.mU.fA.mU.fU.mC.fU.mC.fA.mG.fU. |
| | | | mG.fA |
| SEQ ID | 45 | AP336AS/PCS53 | PmU.fA.mC.fA.mA.fA.mA.fG.mC.fA.mA.fA.mA.fC.mA.fG. |
| NO: 105 | | | mG.fU.mC.fC.mA.fG.mU.fA.mU.fU.mC.fU.mC.fA.mG.fU. |
| | | | mG.fA |
| SEQ ID | 46 | AP336AS/PCS29 | PmU.fG.mC.fA.mA.fA.mA.fC.mA.fG.mG.fU.mC.fU.mA.fG. |
| NO: 106 | | | mA.fA.mA.fC.mA.fG.mU.fA.mU.fU.mC.fU.mC.fA.mG.fU. |
| | | | mG.fA |
| SEQ ID | 47 | AP336AS/PCS57 | PmU.fC.mA.fA.mA.fA.mG.fC.mA.fA.mA.fA.mC.fA.mG.fG. |
| NO: 107 | | | mU.fC.mU.fC.mA.fG.mU.fA.mU.fU.mC.fU.mC.fA.mG.fU. |
| | | | mG.fA |
| SEQ ID | 48 | AP336AS/PCS52 | PmU.fA.mG.fA.mA.fU.mA.fA.mA.fU.mA.fU.mC.fU.mU.fC. |
| NO: 108 | | | mA.fA.mG.fC.mA.fG.mU.fA.mU.fU.mC.fU.mC.fA.mG.fU. |
| | | | mG.fA |
| SEQ ID | 49 | AP336AS/PCS55 | PmU.fA.mA.fA.mG.fC.mA.fA.mA.fA.mC.fA.mG.fG.mU.fC. |
| NO: 109 | | | mU.fA.mG.fC.mA.fG.mU.fA.mU.fU.mC.fU.mC.fA.mG.fU. |
| | | | mG.fA |
| SEQ ID | 50 | AP277/PCS29 | [mU][#][fG][#][mC][fA][mA][fA][mA][fC][mA][fG][mG] |
| NO: 110 | | | [fU][mC][fU][#][mA][#][fG][#][mA][#][fA][#][rA][mA][#] |
| | | | [fC][#][mC][fU][mG][fC][mC][fU][mA][fU][mC][mC][#] |
| | | | [mA][#][mA][#][3XGalNac] |
| SEQ ID | 51 | AP277/PCS52 | [mU][#][fA][#][mG][fA][mA][fU][mA][fA][mA][fU][mA] |
| NO: 111 | | | [fU][mC][fU][#][mU][#][fC][#][mA][#][fA][#][rG][mA][#] |
| | | | [fC][#][mC][fU][mG][fC][mC][fU][mA][fU][mC][mC][#] |
| | | | [mA][#][mA][#][3XGalNac] |
| SEQ ID | 52 | AP28/PCS48 | [mU][#][fG][#][mU][fG][mA][fC][mA][fC][mA][fA][mA] |
| NO: 112 | | | [fG][mC][fA][#][mG][#][fG][#][mU][#][fG][#][rC][mG][#] |
| | | | [fU][#][mA][fC][mU][fC][mC][fU][mU][fG][mU][mu][#] |
| | | | [mG][#][mA][#][3XGalNAc] |
| SEQ ID | 53 | AP277/PCS48 | [mU][#][fG][#][mU][fG][mA][fC][mA][fC][mA][fA][mA] |
| NO: 113 | | | [fG][mC][fA][#][mG][#][fG][#][mU][#][fG][#][rC][mA][#] |
| | | | [fC][#][mC][fU][mG][fC][mC][fU][mA][fU][mC][mC][#] |
| | | | [mA][#][mA][#][3XGalNac] |
| SEQ ID | 54 | AP337/PCS44 | [mU][#][fC][#][mU][fU][mC][fA][mA][fG][mU][fU][mA] |
| NO: 114 | | | [fC][#][mA][#][fA][#][mA][#][fA][#][mG][#][fC][#][rA][mG] |
| | | | [#][fU][#][mA][fU][mU][fC][mU][fC][mA][fG][mU][fG][#] |
| | | | [mC][#][fA][#][3xGalNac] |

Table 5b shows linked second and third nucleic acid portions of the molecules. Linking is direct to give rise to a single contiguous strand.

| | | | |
|---|---|---|---|
| SEQ ID | 1 | AP277AS/PCS44 | PmU.fU.mG.fG.mA.fU.mA.fG.mG.fC.mA.fG.mG.fU.mG.fG. |
| NO: 115 | | | mA.fC.mU.fU.mU.fU.mG.fU.mA.fA.mC.fU.mU.fG.mA.fA. |
| | | | mG.fA |
| SEQ ID | 2 | AP277AS/PCS46 | PmU.fU.mG.fG.mA.fU.mA.fG.mG.fC.mA.fG.mG.fU.mG.fG. |
| NO: 116 | | | mA.fC.mU.fC.mC.fU.mG.fU.mU.fU.mU.fG.mC.fU.mU.fU. |
| | | | mU.fA |
| SEQ ID | 3 | AP277AS/PCS53 | PmU.fU.mG.fG.mA.fU.mA.fG.mG.fC.mA.fG.mG.fU.mG.fG. |
| NO: 117 | | | mA.fC.mU.fU.mG.fU.mU.fU.mU.fG.mC.fU.mU.fU.mU.fG. |
| | | | mU.fA |
| SEQ ID | 4 | AP277AS/PCS29 | PmU.fU.mG.fG.mA.fU.mA.fG.mG.fC.mA.fG.mG.fU.mG.fG. |
| NO: 118 | | | mA.fC.mU.fU.mA.fG.mA.fC.mC.fU.mG.fU.mU.fU.mU.fG. |
| | | | mC.fA |
| SEQ ID | 5 | AP277AS/PCS57 | PmU.fU.mG.fG.mA.fU.mA.fG.mG.fC.mA.fG.mG.fU.mG.fG. |
| NO: 119 | | | mA.fC.mU.fC.mU.fG.mU.fU.mU.fU.mG.fC.mU.fU.mU.fU. |
| | | | mG.fA |
| SEQ ID | 6 | AP277AS/PCS52 | PmU.fU.mG.fG.mA.fU.mA.fG.mG.fC.mA.fG.mG.fU.mG.fG. |
| NO: 120 | | | mA.fC.mU.fA.mA.fG.mA.fU.mA.fU.mU.fU.mA.fU.mU.fC. |
| | | | mU.fA |
| SEQ ID | 7 | AP277AS/PCS55 | PmU.fU.mG.fG.mA.fU.mA.fG.mG.fC.mA.fG.mG.fU.mG.fG. |
| NO: 121 | | | mA.fC.mU.fA.mC.fC.mU.fG.mU.fU.mU.fU.mG.fC.mU.fU. |
| | | | mU.fA |
| SEQ ID | 8 | AP377As/PCS44 | PmU.fG.mC.fA.mC.fU.mG.fA.mG.fA.mA.fU.mA.fC.mU.fG. |
| NO: 122 | | | mU.fC.mC.fU.mU.fU.mG.fU.mA.fA.mC.fU.mU.fG.mA.fA. |
| | | | mG.fA |
| SEQ ID | 9 | AP377As/PCS46 | PmU.fG.mC.fA.mC.fU.mG.fA.mG.fA.mA.fU.mA.fC.mU.fG. |
| NO: 123 | | | mU.fC.mC.fC.mC.fU.mG.fU.mU.fU.mU.fG.mC.fU.mU.fU. |
| | | | mU.fA |
| SEQ ID | 10 | AP377As/PCS53 | PmU.fG.mC.fA.mC.fU.mG.fA.mG.fA.mA.fU.mA.fC.mU.fG. |
| NO: 124 | | | mU.fC.mC.fU.mG.fU.mU.fU.mU.fG.mC.fU.mU.fU.mU.fG. |
| | | | mU.fA |
| SEQ ID | 11 | AP377As/PCS29 | PmU.fG.mC.fA.mC.fU.mG.fA.mG.fA.mA.fU.mA.fC.mU.fG. |
| NO: 125 | | | mU.fC.mC.fU.mA.fG.mA.fC.mC.fU.mG.fU.mU.fU.mU.fG. |
| | | | mC.fA |
| SEQ ID | 12 | AP377As/PCS57 | PmU.fG.mC.fA.mC.fU.mG.fA.mG.fA.mA.fU.mA.fC.mU.fG. |
| NO: 126 | | | mU.fC.mC.fC.mU.fG.mU.fU.mU.fU.mG.fC.mU.fU.mU.fU. |
| | | | mG.fA |
| SEQ ID | 13 | AP377As/PCS52 | PmU.fG.mC.fA.mC.fU.mG.fA.mG.fA.mA.fU.mA.fC.mU.fG. |
| NO: 127 | | | mU.fC.mC.fA.mA.fG.mA.fU.mA.fU.mU.fU.mA.fU.mU.fC. |
| | | | mU.fA |
| SEQ ID | 14 | AP377As/PCS55 | PmU.fG.mC.fA.mC.fU.mG.fA.mG.fA.mA.fU.mA.fC.mU.fG. |
| NO: 128 | | | mU.fC.mC.fA.mC.fC.mU.fG.mU.fU.mU.fU.mG.fC.mU.fU. |
| | | | mU.fA |
| SEQ ID | 15 | AP028As/PCS44 | PmU.fC.mA.fA.mC.fA.mA.fG.mG.fA.mG.fU.mA.fC.mC.fC. |
| NO: 129 | | | mG.fG.mG.fU.mU.fU.mG.fU.mA.fA.mC.fU.mU.fG.mA.fA. |
| | | | mG.fA |
| SEQ ID | 16 | AP028As/PCS46 | PmU.fC.mA.fA.mC.fA.mA.fG.mG.fA.mG.fU.mA.fC.mC.fC. |
| NO: 130 | | | mG.fG.mG.fC.mC.fU.mG.fU.mU.fU.mU.fG.mC.fU.mU.fU. |
| | | | mU.fA |
| SEQ ID | 17 | AP028As/PCS53 | PmU.fC.mA.fA.mC.fA.mA.fG.mG.fA.mG.fU.mA.fC.mC.fC. |
| NO: 131 | | | mG.fG.mG.fU.mG.fU.mU.fU.mU.fG.mC.fU.mU.fU.mU.fG. |
| | | | mU.fA |
| SEQ ID | 18 | AP028As/PCS29 | PmU.fC.mA.fA.mC.fA.mA.fG.mG.fA.mG.fU.mA.fC.mC.fC. |
| NO: 132 | | | mG.fG.mG.fU.mA.fG.mA.fC.mC.fU.mG.fU.mU.fU.mU.fG. |
| | | | mC.fA |
| SEQ ID | 19 | AP028As/PCS57 | PmU.fC.mA.fA.mC.fA.mA.fG.mG.fA.mG.fU.mA.fC.mC.fC. |
| NO: 133 | | | mG.fG.mG.fC.mU.fG.mU.fU.mU.fU.mG.fC.mU.fU.mU.fU. |
| | | | mG.fA |
| SEQ ID | 20 | AP028As/PCS52 | PmU.fC.mA.fA.mC.fA.mA.fG.mG.fA.mG.fU.mA.fC.mC.fC. |
| NO: 134 | | | mG.fG.mG.fA.mA.fG.mA.fU.mA.fU.mU.fU.mA.fU.mU.fC. |
| | | | mU.fA |
| SEQ ID | 21 | AP028As/PCS55 | PmU.fC.mA.fA.mC.fA.mA.fG.mG.fA.mG.fU.mA.fC.mC.fC. |
| NO: 135 | | | mG.fG.mG.fA.mC.fC.mU.fG.mU.fU.mU.fU.mG.fC.mU.fU. |
| | | | mU.fA |
| SEQ ID | 22 | AP369As/PCS44 | PmU.fC.mU.fU.mG.fU.mC.fC.mA.fG.mC.fU.mU.fU.mA.fU. |
| NO: 136 | | | mU.fG.mG.fU.mU.fU.mG.fU.mA.fA.mC.fU.mU.fG.mA.fA. |
| | | | mG.fA |
| SEQ ID | 23 | AP369As/PCS46 | PmU.fC.mU.fU.mG.fU.mC.fC.mA.fG.mC.fU.mU.fU.mA.fU. |
| NO: 137 | | | mU.fG.mG.fC.mC.fU.mG.fU.mU.fU.mU.fG.mC.fU.mU.fU. |
| | | | mU.fA |
| SEQ ID | 24 | AP369As/PCS53 | PmU.fC.mU.fU.mG.fU.mC.fC.mA.fG.mC.fU.mU.fU.mA.fU. |
| NO: 138 | | | mU.fG.mG.fU.mG.fU.mU.fU.mU.fG.mC.fU.mU.fU.mU.fG. |
| | | | mU.fA |
| SEQ ID | 25 | AP369As/PCS29 | PmU.fC.mU.fU.mG.fU.mC.fC.mA.fG.mC.fU.mU.fU.mA.fU. |
| NO: 139 | | | mU.fG.mG.fU.mA.fG.mA.fC.mC.fU.mG.fU.mU.fU.mU.fG. |
| | | | mC.fA |
| SEQ ID | 26 | AP369As/PCS57 | PmU.fC.mU.fU.mG.fU.mC.fC.mA.fG.mC.fU.mU.fU.mA.fU. |
| NO: 140 | | | mU.fG.mG.fC.mU.fG.mU.fU.mU.fU.mG.fC.mU.fU.mU.fU. |
| | | | mG.fA |
| SEQ ID | 27 | AP369As/PCS52 | PmU.fC.mU.fU.mG.fU.mC.fC.mA.fG.mC.fU.mU.fU.mA.fU. |
| NO: 141 | | | mU.fG.mG.fA.mA.fG.mA.fU.mA.fU.mU.fU.mA.fU.mU.fC. |
| | | | mU.fA |
| SEQ ID | 28 | AP369As/PCS55 | PmU.fC.mU.fU.mG.fU.mC.fC.mA.fG.mC.fU.mU.fU.mA.fU. |
| NO: 142 | | | mU.fG.mG.fA.mC.fC.mU.fG.mU.fU.mU.fU.mG.fC.mU.fU. |
| | | | mU.fA |
| SEQ ID | 29 | AP366AS/PCS44 | PmU.fU.mC.fC.mA.fG.mC.fU.mU.fU.mA.fU.mU.fG.mG.fG. |
| NO: 143 | | | mA.fG.mG.fU.mU.fU.mG.fU.mA.fA.mC.fU.mU.fG.mA.fA. |
| | | | mG.fA |
| SEQ ID | 30 | AP366AS/PCS46 | PmU.fU.mC.fC.mA.fG.mC.fU.mU.fU.mA.fU.mU.fG.mG.fG. |
| NO: 144 | | | mA.fG.mG.fC.mC.fU.mG.fU.mU.fU.mU.fG.mC.fU.mU.fU. |
| | | | mU.fA |
| SEQ ID | 31 | AP366AS/PCS53 | PmU.fU.mC.fC.mA.fG.mC.fU.mU.fU.mA.fU.mU.fG.mG.fG. |
| NO: 145 | | | mA.fG.mG.fU.mG.fU.mU.fU.mU.fG.mC.fU.mU.fU.mU.fG. |
| | | | mU.fA |
| SEQ ID | 32 | AP366AS/PCS29 | PmU.fU.mC.fC.mA.fG.mC.fU.mU.fU.mA.fU.mU.fG.mG.fG. |
| NO: 146 | | | mA.fG.mG.fU.mA.fG.mA.fC.mC.fU.mG.fU.mU.fU.mU.fG. |
| | | | mC.fA |
| SEQ ID | 33 | AP366AS/PCS57 | PmU.fU.mC.fC.mA.fG.mC.fU.mU.fU.mA.fU.mU.fG.mG.fG. |
| NO: 147 | | | mA.fG.mG.fC.mU.fG.mU.fU.mU.fU.mG.fC.mU.fU.mU.fU. |
| | | | mG.fA |
| SEQ ID | 34 | AP366AS/PCS52 | PmU.fU.mC.fC.mA.fG.mC.fU.mU.fU.mA.fU.mU.fG.mG.fG. |
| NO: 148 | | | mA.fG.mG.fA.mA.fG.mA.fU.mA.fU.mU.fU.mA.fU.mU.fC. |
| | | | mU.fA |
| SEQ ID | 35 | AP366AS/PCS55 | PmU.fU.mC.fC.mA.fG.mC.fU.mU.fU.mA.fU.mU.fG.mG.fG. |
| NO: 149 | | | mA.fG.mG.fA.mC.fC.mU.fG.mU.fU.mU.fU.mG.fC.mU.fU. |
| | | | mU.fA |
| SEQ ID | 36 | AP367As/PCS44 | PmU.fG.mU.fC.mC.fA.mG.fC.mU.fU.mU.fA.mU.fU.mG.fG. |
| NO: 150 | | | mG.fA.mG.fU.mU.fU.mG.fU.mA.fA.mC.fU.mU.fG.mA.fA. |
| | | | mG.fA |
| SEQ ID | 37 | AP367As/PCS46 | PmU.fG.mU.fC.mC.fA.mG.fC.mU.fU.mU.fA.mU.fU.mG.fG. |
| NO: 151 | | | mG.fA.mG.fC.mC.fU.mG.fU.mU.fU.mU.fG.mC.fU.mU.fU. |
| | | | mU.fA |
| SEQ ID | 38 | AP367As/PCS53 | PmU.fG.mU.fC.mC.fA.mG.fC.mU.fU.mU.fA.mU.fU.mG.fG |
| NO: 152 | | | mG.fA.mG.fU.mG.fU.mU.fU.mU.fG.mC.fU.mU.fU.mU.fG. |
| | | | mU.fA |
| SEQ ID | 39 | AP367As/PCS29 | PmU.fG.mU.fC.mC.fA.mG.fC.mU.fU.mU.fA.mU.fU.mG.fG. |
| NO: 153 | | | mG.fA.mG.fU.mA.fG.mA.fC.mC.fU.mG.fU.mU.fU.mU.fG. |
| | | | mC.fA |
| SEQ ID | 40 | AP367As/PCS57 | PmU.fG.mU.fC.mC.fA.mG.fC.mU.fU.mU.fA.mU.fU.mG.fG. |
| NO: 154 | | | mG.fA.mG.fC.mU.fG.mU.fU.mU.fU.mG.fC.mU.fU.mU.fU. |
| | | | mG.fA |
| SEQ ID | 41 | AP367As/PCS52 | PmU.fG.mU.fC.mC.fA.mG.fC.mU.fU.mU.fA.mU.fU.mG.fG. |
| NO: 155 | | | mG.fA.mG.fA.mA.fG.mA.fU.mA.fU.mU.fU.mA.fU.mU.fC. |
| | | | mU.fA |
| SEQ ID | 42 | AP367As/PCS55 | PmU.fG.mU.fC.mC.fA.mG.fC.mU.fU.mU.fA.mU.fU.mG.fG. |
| NO: 156 | | | mG.fA.mG.fA.mC.fC.mU.fG.mU.fU.mU.fU.mG.fC.mU.fU. |
| | | | mU.fA |
| SEQ ID | 43 | AP336AS/PCS44 | PmU.fC.mA.fC.mU.fG.mA.fG.mA.fA.mU.fA.mC.fU.mG.fU. |
| NO: 157 | | | mC.fC.mC.fU.mU.fU.mG.fU.mA.fA.mC.fU.mU.fG.mA.fA. |
| | | | mG.fA |
| SEQ ID | 44 | AP336AS/PCS46 | PmU.fC.mA.fC.mU.fG.mA.fG.mA.fA.mU.fA.mC.fU.mG.fU. |
| NO: 158 | | | mC.fC.mC.fC.mC.fU.mG.fU.mU.fU.mU.fG.mC.fU.mU.fU. |
| | | | mU.fA |
| SEQ ID | 45 | AP336AS/PCS53 | PmU.fC.mA.fC.mU.fG.mA.fG.mA.fA.mU.fA.mC.fU.mG.fU. |
| NO: 159 | | | mC.fC.mC.fU.mG.fU.mU.fU.mU.fG.mC.fU.mU.fU.mU.fG. |
| | | | mU.fA |
| SEQ ID | 46 | AP336AS/PCS29 | PmU.fC.mA.fC.mU.fG.mA.fG.mA.fA.mU.fA.mC.fU.mG.fU. |
| NO: 160 | | | mC.fC.mC.fU.mA.fG.mA.fC.mC.fU.mG.fU.mU.fU.mU.fG. |
| | | | mC.fA |
| SEQ ID | 47 | AP336AS/PCS57 | PmU.fC.mA.fC.mU.fG.mA.fG.mA.fA.mU.fA.mC.fU.mG.fU. |
| NO: 161 | | | mC.fC.mC.fC.mU.fG.mU.fU.mU.fU.mG.fC.mU.fU.mU.fU. |
| | | | mG.fA |
| SEQ ID | 48 | AP336AS/PCS52 | PmU.fC.mA.fC.mU.fG.mA.fG.mA.fA.mU.fA.mC.fU.mG.fU. |
| NO: 162 | | | mC.fC.mC.fA.mA.fG.mA.fU.mA.fU.mU.fU.mA.fU.mU.fC. |
| | | | mU.fA |
| SEQ ID | 49 | AP336AS/PCS55 | PmU.fC.mA.fC.mU.fG.mA.fG.mA.fA.mU.fA.mC.fU.mG.fU. |
| NO: 163 | | | mC.fC.mC.fA.mC.fC.mU.fG.mU.fU.mU.fU.mG.fC.mU.fU. |
| | | | mU.fA |
| SEQ ID | 50 | AP277/PCS29 | [mU][#][U][#][mG][fG][mA][fU][mA][fG][mG][fC][mA] |
| NO: 164 | | | [fG][mG][f][#][mG][#][fG][#][mA][#][fC][#][rU][mA][#] |
| | | | [fG][#][mA][fC][mC][fU][mG][fU][mU][fU][mU][mG][#] |
| | | | [mC][#][mA][#][3XGalNAc] |
| SEQ ID | 51 | AP277/PCS52 | [mU][#][fU][#][mG][fG][mA][fU][mA][fG][mG][fC][mA] |
| NO: 165 | | | [fG][mG][fU][#][mG][#][fG][#][mA][#][fC][#][rU][mA][#] |
| | | | [fG][#][mA][fU][mA][fU][mU][fU][mA][fU][mU][mC][#] |
| | | | [mU][#][mA][#][3XGalNac] |
| SEQ ID | 52 | AP28/PCS48 | [mU][#][fC][#][mA][fA][mC][fA][mA][fG][mG][fA][mG] |
| NO: 166 | | | [fU][mA][fC][#][mC][#][fC][#][mG][#][fG][#][rG][mU][#] |
| | | | [fG][#][mC][fU][mU][fU][mG][fU][mG][fU][mC][mA][#] |
| | | | [mC][#][mA][#][3XGalNAc] |
| SEQ ID | 53 | AP277/PCS48 | [mU][#][fU][#][mG][fG][mA][fU][mA][fG][mG][fC][mA] |
| NO: 167 | | | [fG][mG][fU][#][mG][#][fG][#][mA][#][fC][#][rU][mU][#] |
| | | | [fG][#][mC][fU][mU][fU][mG][fU][mG][fU][mC][mA][#] |
| | | | [mC][#][mA][#][3XGalNAc] |
| SEQ ID | 54 | AP337/PCS44 | [mU][#][fG][#][mC][fA][mC][fU][mG][fA][mG][fA][mA] |
| NO: 168 | | | [fU][#][mA][#][fC][#][mU][#][fG][#][mU][#][fC][#][rC][mU] |
| | | | [#][fU][mG][fU][mA][fA][mC][fU][mU][fG][mA][fA][#] |
| | | | [mG][#][fA][#][3xGalNac] |

While the methods are shown and described as being a series of acts that are performed in a specific sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the Examples described above may be combined with aspects of any of the other Examples described to form further Examples.

It will be understood that the above descriptions of particular embodiments are provided by way of example only and that various modifications may be made by those skilled in the art. What has been described above includes Examples of one or more embodiments. It is, of course, not possible to describe every conceivable modification and alteration of the above compounds, compositions or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

### EXAMPLES

The following Examples illustrate certain embodiments of the present disclosure and are not limiting. Moreover, where specific embodiments are provided, the inventors have contemplated generic application of those specific embodiments. For example, disclosure of a construct having a specific motif or modification patterns provides reasonable support for additional constructs having the same or similar motif or modification patterns.

The syntheses of the RNAi constructs, e.g., muRNA constructs, disclosed herein have been carried out using synthesis methods known to the person skilled in the art, such as synthesis methods disclosed in https://en.wikipedia.org/wiki/Oligonucleotide_synthesis {retrieved on 16 Feb. 2022}, where the methods disclosed on this website are incorporated by reference herein in their entirety. The only difference to the synthesis method disclosed in this reference is that GalNAc phosphoramidite immobilized on a support is used in the synthesis method during the first synthesis step.

### Example 1

Materials and Methods

Cell Culture:

HepG2 (ATCC cat. 85011430) cells were maintained by biweekly passing in EMEM supplemented with 10% FBS, 20 mM L-glutamine, 10 mM HEPES pH 7.2, 1 mM sodium pyruvate, 1×MEM non-essential amino acids, and 1× Pen/Strep (EMEM complete).

PCSK9 and APOC3 Combo identification and compound preparation: Combination compounds with the best performing PCSK9 and APOC3 sequences were designed and synthesized as 49 candidates; see Table 5 above. Compounds were dissolved to 50 uM in molecular biology grade water and annealed by heating at 95 C for 5 minutes followed by gradual cooling to room temperature.

APOC3/PCSK9 Combo—Screen: Based on data from screens with PCSK9 and APOC3 duplexes, the best performing APOC3 and PCSK9 sequences were combined into 49 candidates and tested (combinations of numbers 1 to 49 of Tables 5a and 5b respectively, where constructs numbered equally have been combined) in dose curves. HepG2 cells were collected by trypsinization and seeded in 96 well tissue culture plates at 10,000 cells per well in 50 uL complete EMEM with 20% FBS and allowed to rest for 4 hours. Transfection complexes were formed by gently mixing 36 pmoles of each duplex in 180 uL OptiMEM with 2.16 uL RNAiMax in 180 uL OptiMEM to make 360 uL total complex. A two fold dilution series was then performed with basal OptiMEM. 50 uL of each dilution was added to respective triplicates of HepG2 cells to make a final dilution series of 50 nM down to 0.32 nM in a volume of 100 uL, 50/50 EMEM/OptiMEM at 10% FBS. 72 hours post transfection, cells were harvested and RNA isolated using the PureLink Pro 96 total RNA Purification Kit (ThermoFisher, 12173011A) according to the manufacturer protocol. Harvested RNA was assayed for both PCSK9 and APOC3 expression via Taqman qPCR using the Luna Universal Probe One-Step RT-qPCR Kit (NEB, E3006). Two separate qPCR assays were performed for each sample using an either an APOC3 Taqman probe (Hs00906501_g1-FAM) or PCSK9 probe (Hs00906501_g1-FAM) multiplexed with a common GAPDH VIC probe (ThermoFisher, 4326317E). Thermocycling and data acquisition was performed with an Applied Biosystems QuantStudio 3 Real-Time PCR System.

Results

Tables 6a and 6b below shows IC50 values (in nM) for specific constructs selected in accordance with the Examples. Table 6a shows PCSK9 knock-down, and Table 6b shows APOC3 knock-down. The numbers after “SR1-” in the first column correspond to the construct numbering in Tables 5a and 5b above.

TABLE 6a

| | | % KD at |
|---|---|---|
| Sequence | | highest Conc |
| ID | IC50 | (50 nM) |
| SR1-25 | 3.14 | 92 |
| SR1-26 | 5.07 | 85 |
| SR1-27 | 5.24 | 86 |
| SR1-31 | 5.56 | 91 |
| SR1-28 | 6.04 | 84 |
| SR1-24 | 5.85 | 84 |
| SR1-45 | 6.08 | 81 |
| SR1-22 | 6.37 | 83 |
| SR1-40 | 6.57 | 83 |
| SR1-47 | 6.59 | 81 |
| SR1-39 | 6.65 | 86 |
| SR1-32 | 7.31 | 86 |
| SR1-36 | 7.50 | 83 |
| SR1-18 | 7.57 | 86 |
| SR1-19 | 7.78 | 85 |
| SR1-49 | 8.13 | 76 |
| SR1-46 | 8.40 | 77 |
| SR1-38 | 8.45 | 85 |
| SR1-21 | 8.56 | 74 |
| SR1-23 | 8.60 | 74 |
| SR1-4 | 8.83 | 84 |
| SR1-34 | 8.86 | 86 |
| SR1-29 | 9.16 | 86 |
| SR1-17 | 9.27 | 78 |
| SR1-15 | 9.31 | 81 |
| SR1-37 | 9.58 | 75 |
| SR1-48 | 10.18 | 78 |
| SR1-1 | 10.23 | 80 |
| SR1-7 | 10.36 | 79 |
| SR1-33 | 10.37 | 83 |
| SR1-20 | 10.55 | 80 |
| SR1-35 | 10.79 | 78 |
| SR1-9 | 10.93 | 75 |
| SR1-3 | 12.03 | 78 |
| SR1-5 | 12.60 | 78 |
| SR1-2 | 14.01 | 76 |
| SR1-11 | 15.74 | 62 |
| SR1-16 | 16.02 | 73 |
| SR1-42 | 16.04 | 68 |
| SR1-41 | 16.76 | 81 |
| SR1-30 | 17.95 | 81 |
| SR1-6 | 18.14 | 73 |
| SR1-13 | 18.99 | 64 |
| SR1-43 | 19.41 | 69 |
| SR1-44 | 19.98 | 73 |
| SR1-10 | 21.05 | 64 |
| SR1-8 | 23.20 | 44 |
| SR1-14 | 24.16 | 44 |
| SR1-12 | 30.19 | 46 |

TABLE 6b

| | | % KD at |
|---|---|---|
| Sequence | | highest |
| ID | IC50 | Conc (50nM) |
| SR1-36 | 3.75 | 93 |
| SR1-25 | 4.01 | 93 |
| SR1-20 | 4.04 | 79 |
| SR1-40 | 4.44 | 93 |
| SR1-34 | 4.53 | 88 |
| SR1-4 | 4.80 | 84 |
| SR1-1 | 5.15 | 85 |
| SR1-26 | 5.23 | 88 |
| SR1-27 | 5.52 | 89 |
| SR1-45 | 5.81 | 93 |
| SR1-18 | 6.94 | 78 |
| SR1-19 | 6.97 | 70 |
| SR1-31 | 7.04 | 93 |
| SR1-35 | 7.59 | 79 |
| SR1-38 | 7.69 | 90 |
| SR1-24 | 7.86 | 81 |
| SR1-42 | 8.00 | 91 |
| SR1-47 | 8.13 | 88 |
| SR1-39 | 8.80 | 84 |
| SR1-29 | 8.87 | 88 |
| SR1-41 | 8.94 | 86 |
| SR1-49 | 8.94 | 83 |
| SR1-3 | 9.04 | 79 |
| SR1-46 | 9.11 | 84 |
| SR1-48 | 9.62 | 86 |
| SR1-2 | 10.14 | 76 |
| SR1-7 | 10.42 | 78 |
| SR1-44 | 10.45 | 87 |
| SR1-28 | 10.49 | 83 |
| SR1-22 | 10.72 | 80 |
| SR1-43 | 10.99 | 84 |
| SR1-37 | 11.43 | 81 |
| SR1-23 | 11.85 | 77 |
| SR1-32 | 11.96 | 80 |
| SR1-5 | 12.29 | 74 |
| SR1-21 | 13.94 | 71 |
| SR1-13 | 15.73 | 60 |
| SR1-8 | 15.90 | 63 |
| SR1-15 | 16.95 | 63 |
| SR1-30 | 17.59 | 83 |
| SR1-9 | 18.21 | 71 |
| SR1-6 | 18.70 | 69 |
| SR1-12 | 19.50 | 59 |
| SR1-17 | 20.07 | 48 |
| SR1-10 | 21.18 | 71 |
| SR1-16 | 23.41 | 61 |
| SR1-11 | 23.98 | 64 |
| SR1-33 | 33.86 | 49 |
| SR1-14 | 52.14 | 32 |

The IC₅₀ data in the single- to double-digit nanomolar range demonstrate outstanding performance of numerous constructs as described herein. Of note, either target of the double-targeting constructs is knocked down by each of a multitude of constructs.

### Example 2

Materials and Methods

Cell Culture:

Human primary hepatocytes (5 donor pooled—Sekisui XenoTech, HPCH05+) were thawed immediately prior to experimentation and cultured in 1× complete Williams medium (Gibco, A1217601) supplemented with Hepatocytes plating supplement pack (Gibco, CM3000). FBS concentration was modified from manufacture recipe to a final 2.5% (as opposed to 5%) for compound stability.

Identification and compound preparation for PCSK9 and APOC3 Combo containing GalNAc: Combination compounds with PCSK9 and APOC3 sequences were designed and synthesized. In some combination, the bulge in the central part of the molecule has 5 nucleotides in each strand (see Table 7a) whereas in other combination, the bulge in the central part of the molecule has 4 nucleotides (see Table 7b). Compounds were dissolved to 50 uM in molecular biology grade water and annealed by heating at 95° C. for 5 minutes followed by gradual cooling to room temperature.

TABLE 7a
GalNAc-containing muRNAs for ApoC3-PCSK9 with 5 nucleotides in the bulge
region, where the corresponding strands “S + AS”, e.g., Construct ID Nos. 1 + 2,
constitute an muRNA construct.

| SEQ ID | Construct | | | |
|---|---|---|---|---|
| NO: | ID No. | muRNA | Strand | Sequence |
| 169 | 1 | P29-A28 | S | [mU][#][fG][#][mC][fA][mA][fA][mA][fC] |
| | | | | [mA][fG][mG][fU][mC][fU][#][mA][#][fG] |
| | | | | [#][mA][#][A][#][rA][mG][#][fU][#][mA] |
| | | | | [fC][mU][fC][mC][fU][mU][fG][mU][mU][#] |
| | | | | [mG][#][mA][#][3XGalNAc] |
| 170 | 2 | P29-A28 | AS | [mU][#][fC][#][mA][fA][mC][fA][mA][fG] |
| | | | | [mG][fA][mG][fU][mA][fC][#][mC][#][fC] |
| | | | | [#][mG][#][fG][#][fG][mA][#][fG][#][mA] |
| | | | | [fC][mC][fU][mG][fU][mU][fU][mU][mG][#] |
| | | | | [mC][#][mA][#][3XGalNAc] |
| 110 | 3 | P29-A277 | S | [mU][#][fG][#][mC][fA][mA][fA][mA][fC] |
| | | | | [mA][fG][mG][fU][mC][fU][#][mA][#][fG] |
| | | | | [#][mA][#][fA][#][rA][mA][#][fC][#][mC] |
| | | | | [fU][mG][fC][mC][fU][mA][fU][mC][mC][#] |
| | | | | [mA][#][mA][#][3XGalNac] |
| 164 | 4 | P29-A277 | AS | [mU][#][fU][#][mG][fG][mA][fU][mA][fG] |
| | | | | [mG][fC][mA][fG][mG][fU][#][mG][#][fG] |
| | | | | [#][mA][#][fC][#][rU][mA][#][fG][#][mA] |
| | | | | [fC][mC][fU][mG][fU][mU][fU][mU][mG][#] |
| | | | | [mC][#][mA][#][3XGalNAc] |
| 171 | 5 | P52-A28 | S | [mU][#][fA][#][mG][fA][mA][fU][mA][fA] |
| | | | | [mA][fU][mA][fU][mC][fU][#][mU][#][fC] |
| | | | | [#][mA][#][fA][#][rG][mG][#][fU][#][mA] |
| | | | | [fC][mU][fC][mC][fU][mU][fG][mU][mU][#] |
| | | | | [mG][#][mA][#][3XGalNac] |
| 172 | 6 | P52-A28 | AS | [mU][#][fC][#][mA][fA][mC][fA][mA][fG] |
| | | | | [mG][fA][mG][fU][mA][fC][#][mC][#][fC] |
| | | | | [#][mG][#][fG][#][rG][mA][#][fG][#][mA] |
| | | | | [fU][mA][fU][mU][fU][mA][fU][mU][mC][#] |
| | | | | [mU][#][mA][#][3XGalNac] |
| 111 | 7 | P52-A277 | S | [mU][#][fA][#][mG][fA][mA][fU][mA][fA] |
| | | | | [mA][fU][mA][fU][mC][fU][#][mU][@][fC] |
| | | | | [#][mA][#][fA][#][rG][mA][#][fC][#][mC] |
| | | | | [fU][mG][fC][mC][fU][mA][fU][mC][mC][#] |
| | | | | [mA][#][mA][#][3XGalNac] |
| 165 | 8 | P52-A277 | AS | [mU][#[fU][#][mG][fG][mA][fU][mA][fG] |
| | | | | [mG][fC][mA][fG][mG][fU][#][mG][#][fG] |
| | | | | [#][mA][#][fC][#][rU][mA][#][fG][#][mA] |
| | | | | [fU][mA][fU][mU][fU][mA][fU][mU][mC][#] |
| | | | | [mU][#][mA][#][3XGalNac] |
| 173 | 9 | P57-A277 | S | [mU][#][fC][#][mA][fA][mA][fA][mG][fC] |
| | | | | [mA][fA][mA][fA][mC][fA][#][mG][#][fG] |
| | | | | [#][mU][#][fC][#][rU][mA][#][fC][#][mC] |
| | | | | [fU][mG][fC][mC][fU][mA][fU][mC][mC][#] |
| | | | | [mA][#][mA][#][3XGalNac] |
| 174 | 10 | P57-A277 | AS | [mU][#][fU][#][mG][fG][mA][fU][mA][fG] |
| | | | | [mG][fC][mA][fG][mG][fU][#][mG][#][fG] |
| | | | | [#][mA][#][fC][#][rU][mU][#][fG][#][mU][fU] |
| | | | | [mU][fU][mG][fC][mU][fU][mU][mU][#] |
| | | | | [mG][#][mA][#][3XGalNac] |

TABLE 7b
GalNAc containing muRNAs for ApoC3-PCSK9 with 4 nucleotides in the bulge
region, where the corresponding strands “S + AS”, e.g., Construct ID Nos. 1 + 2, constitute
an muRNA construct. The denotation “(15)” in the muRNA name means that 15
nucleotides are on either side of the bulge within a contiguous strand.

| SEQ ID | Construct | | | |
|---|---|---|---|---|
| NO: | ID No. | muRNA | Strand | Sequence |
| 175 | 11 | P29- | S | [mU][#][fG][#][mC][fA][mA][fA][mA][fC][mA] |
| | | A28(15) | | [fG][mG][fU][mC][fU][#][mA][#][fG][#][mA][#] |
| | | | | [fA][#][rA][fG][#][mG][#][fU][mA][fC][mU][fC] |
| | | | | [mC][fU][mU][fG][mU][mU][#][mG][#][mA][#] |
| | | | | [3XGalNAc] |
| 176 | 12 | P29- | AS | [mU][#][fC][#][mA][fA][mC][fA][mA][fG][mG] |
| | | A28(15) | | [fA][mG][fU][mA][fC][#][mC][#][fC][#][mG][#] |
| | | | | [fG][#][rG][fU][#][mA][#][fG][mA][fC][mC][fU] |
| | | | | [mG][fU][mU][fU][mU][mG][#][mC][#][mA][#] |
| | | | | [3XGalNAc] |
| 177 | 13 | P29- | S | [mU][#][fG][#][mC][fA][mA][fA][mA][fC][mA] |
| | | A277(15) | | [fG][mG][fU][mC][fU][#][mA][#][fG][#][mA][#] |
| | | | | [fA][#][rA][fC][#][mA][#][fC][mC][fU][mG][fC] |
| | | | | [mC][fU][mA][fU][mC][mC][#][mA][#][mA][#] |
| | | | | [3XGalNac] |
| 178 | 14 | P29- | AS | [mU][#][fU][#][mG][fG][mA][fU][mA][fG][mG] |
| | | A277(15) | | [fC][mA][fG][mG][fU][#][mG][#][fG][#][mA][#] |
| | | | | [fC][#][rU][fU][#][mA][#][fG][mA][fC][mC][fU] |
| | | | | [mG][fU][mU][fU][mU][mG][#][mC][#][mA][#] |
| | | | | [3XGalNAc] |
| 179 | 15 | P52- | S | [mU][#][fA][#][mG][fA][mA][fU][mA][fA][mA] |
| | | A28(15) | | [fU][mA][fU][mC][fU][#][mU][#][fC][#][mA][#][fA] |
| | | | | [#][rG][fG][#][mG][#][fU][mA][fC][mU][fC][mC] |
| | | | | [fU][mU][fG][mU][mU][#][mG][#][mA][#] |
| | | | | [3XGalNAc] |
| 180 | 16 | P52- | AS | [mU][#][fC][#][mA][fA][mC][fA][mA][fG][mG] |
| | | A28(15) | | [fA][mG][fU][mA][fC][#][mC][#][fC][#][mG][#][fG] |
| | | | | [#][rG][fA][#][mA][#][fG][mA][fU][mA][fU][mU] |
| | | | | [fU][mA][fU][mU][mC][#][mU][#][mA][#] |
| | | | | [3XGalNac] |
| 181 | 17 | P52- | S | [mU][#][fA][#][mG][fA][mA][fU][mA][fA][mA][fU] |
| | | A277(15) | | [mA][fU][mC][fU][#][mU][#][fC][#][mA][#][fA] |
| | | | | [#][rG][fC][#][mA][#][fC][mC][fU][mG][fC][mC] |
| | | | | [fU][mA][fU][mC][mC][#][mA][#][mA][#] |
| | | | | [3XGalNac] |
| 182 | 18 | P52- | AS | [mU][#][fU][#][mG][fG][mA][fU][mA][fG][mG] |
| | | A277(15) | | [fC][mA][fG][mG][fU][#][mG][#][fG][#][mA][#][fC] |
| | | | | [#][rU][fA][#][mA][#][fG][mA][fU][mA][fU][mU] |
| | | | | [fU][mA][fU][mU][mC][#][mU][#][mA][#] |
| | | | | [3XGalNac] |
| 183 | 19 | P44- | S | [mU][#][fC][#][mU][fU][mC][fA][mA][fG][mU] |
| | | | | [fU][mA][fC][mA][fA][#][mA][#][mA][#][fG][#] |
| | | A28(15) | | [mC][#][rA][fG][#][mG][#][fU][mA][fC][mU][fC] |
| | | | | [mC][fU][mU][fG][mU][mU ][#][mG][#][mA][#] |
| | | | | [3XGalNac] |
| 184 | 20 | p44- | AS | [mU][#][fC][#][mA][fA][mC][fA][mA][fG][mG] |
| | | A28(15) | | [fA][mG][fU][mA][fC][#][mC][#][fC][#][mG][#][fG] |
| | | | | [#][rG][fU][#][mU][#][fU][mG][fU][mA][fA][mC] |
| | | | | [fU][mU][fG][mA][mA][#][mG][#][mA][#] |
| | | | | [3XGalNac] |

APOC3/PCSK9 Combo—Screen:

On the day of transfection, primary human hepatocytes were thawed in 45 mL of human OptiThaw (Sekisui XenoTech, K8000) and centrifuged down at 200 g for 5 minutes. Cells were resuspended in 2× complete WEM and counted. Cells were then plated in 50 μL of 2× complete WEM at 25,000 cells per well on 96 well type 1 rat tail Collagen plates and allowed to rest and attach for four hours before transfection. After rest, the compounds were diluted further to 2 μM in basal WEM. A seven step, five fold dilution series was prepared in basal WEM from 2 μM to 0.000128 μM 50 μL of each 2 μM compound was added to respective triplicates of the plated hepatocytes for a final concentration of 1 μM down to 0.000064 μM in a volume of 100 uL 1× complete WEM.

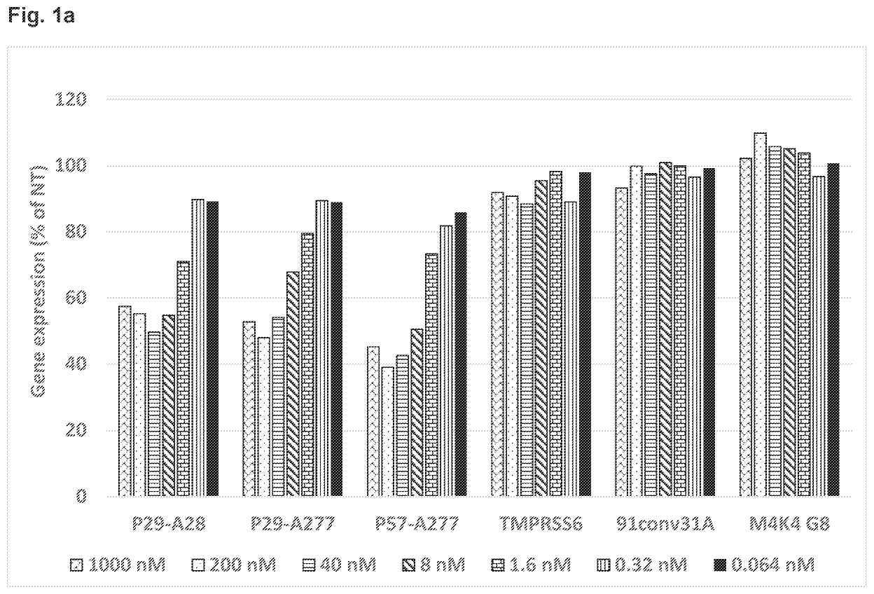

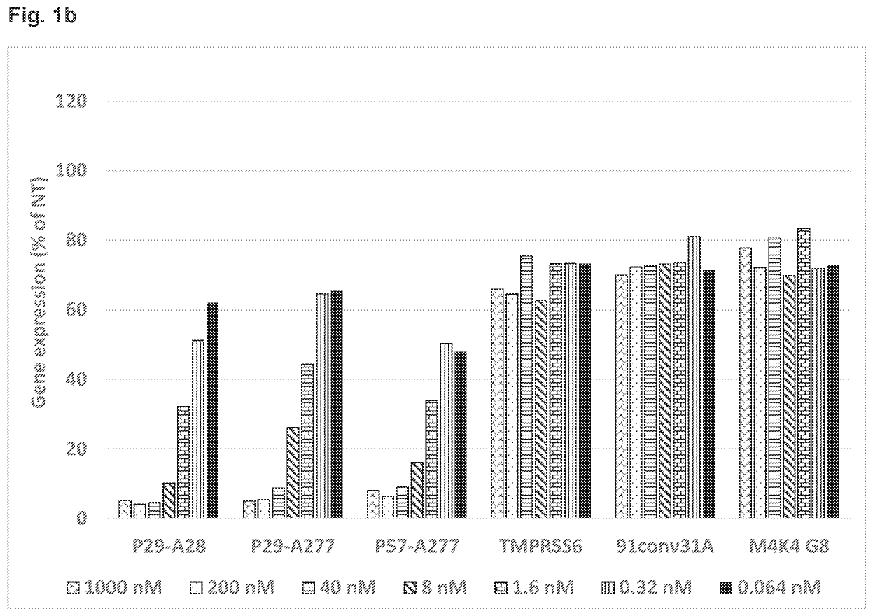

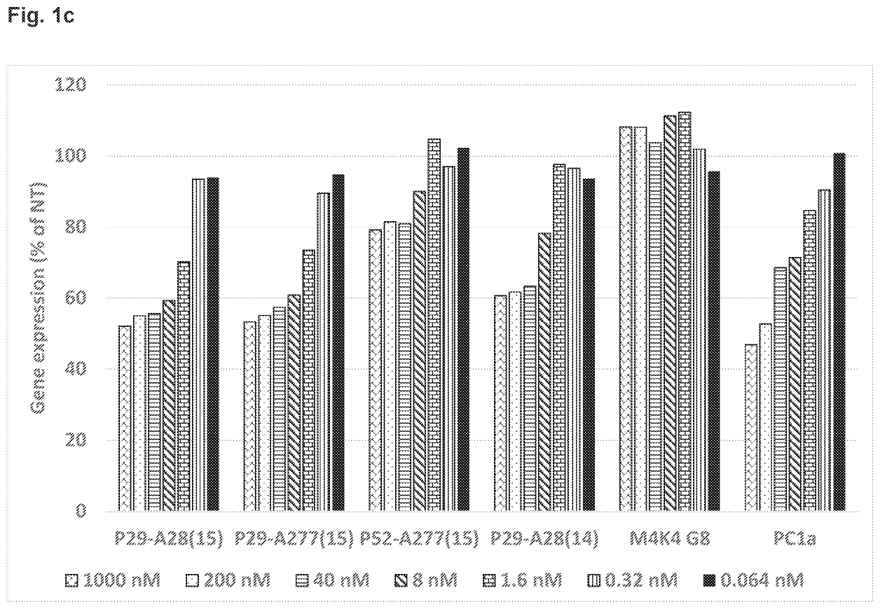

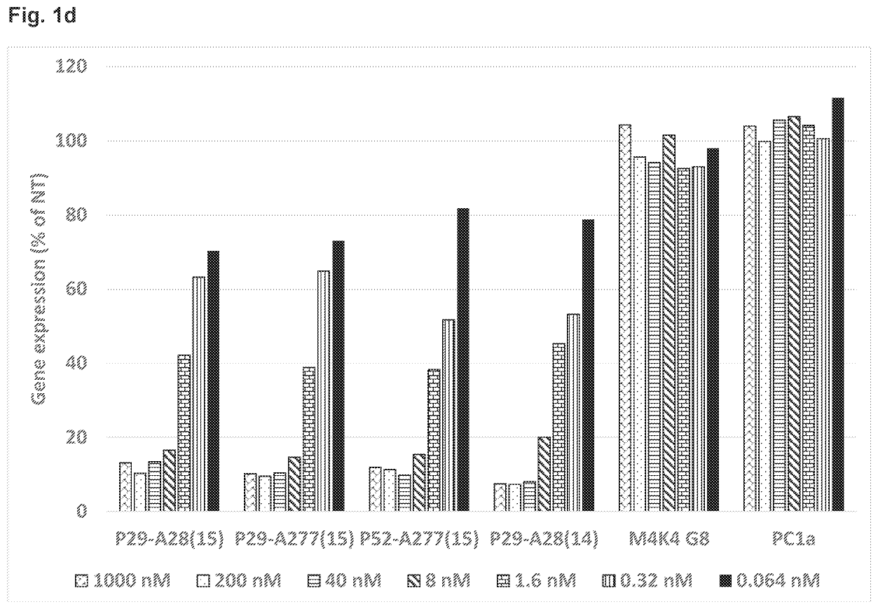

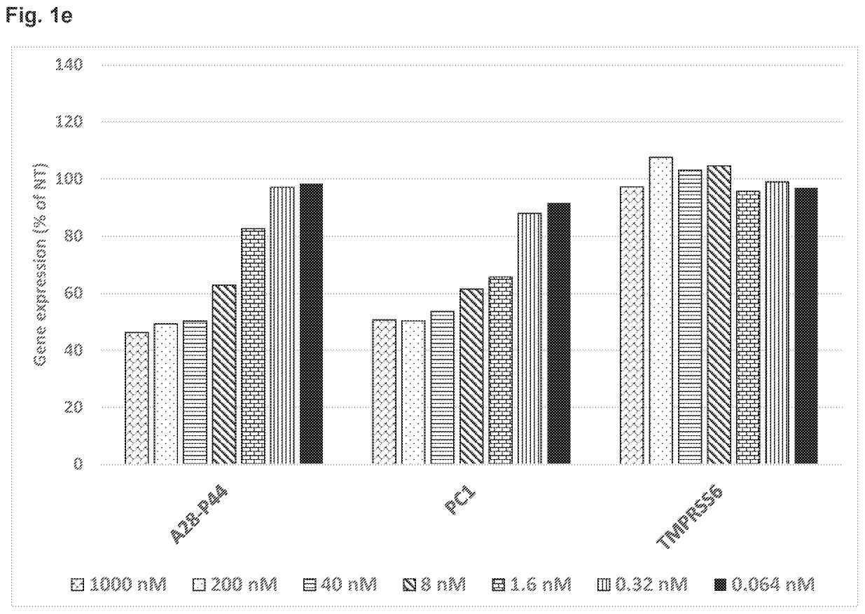

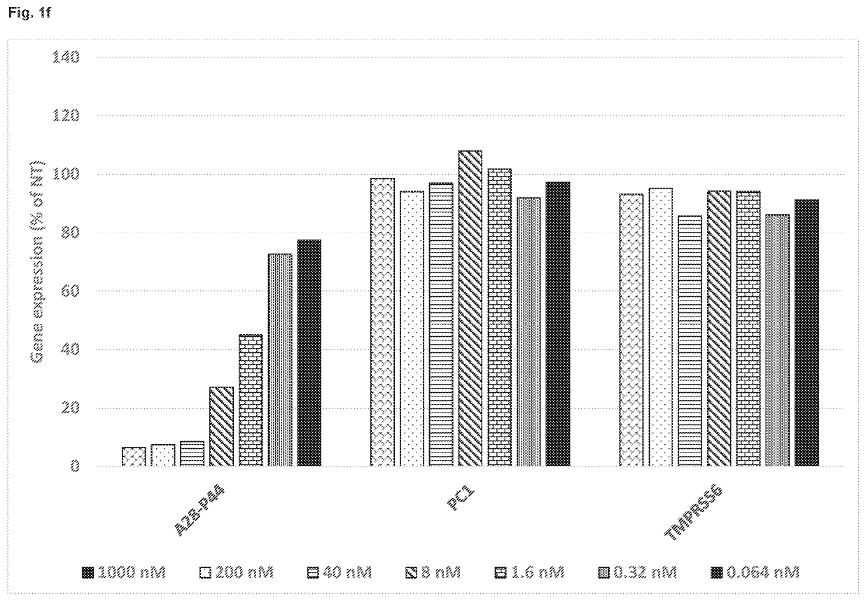

72 hours post transfection, cells were harvested and RNA isolated using the PureLink Pro 96 total RNA Purification Kit (ThermoFisher, 12173011A) according to the manufacturer protocol. Harvested RNA was assayed for both PCSK9 and APOC3 expression via Taqman qPCR using the Luna Universal Probe One-Step RT-qPCR Kit (NEB, E3006). Two separate qPCR assays were performed for each sample using an either an APOC3 Taqman probe (Hs00906501_g1-FAM) or PCSK9 probe (Hs00906501_g1-FAM) multiplexed with a common GAPDH VIC probe (ThermoFisher, 4326317E). Thermocycling and data acquisition was performed with an Applied Biosystems QuantStudio 3 Real-Time PCR System. FIGS. 1a, 1c, and 1e show the dose-response inhibition of PCSK9 mRNA and FIGS. 1b, 1d, and 1f show the dose-response inhibition of APOC3 mRNA

### Example 3

muRNA (APOC3/PCSK9 combination) Leads for Candidate Screening Study in Male UPA HUMANIZED LIVER MICE Mice, non-GLP

1. Study Objective(s)

The objective of this non-GLP study is to:
(i) Evaluate the utility of humanized liver UPA HUMANIZED LIVER MICE mouse model to study performance of muRNA (dual-targeting combination) GalNAc-siRNA molecules.
(ii) Evaluate a dose response effect of three selected muRNA leads for candidate GalNAc-siRNA constructs (with 15 Configuration) targeting APOC3 and PCSK9 using the humanized liver UPA HUMANIZED LIVER MICE mouse model by subcutaneous administration and after 14-day survival.

At necropsy, 3 liver biopsies per animal will be preserved in separate vials in RNAlater, flash frozen, and stored at −80° C. Three more liver biopsies will be taken, flash frozen in the same vial, and stored at −80° C. Harvested tissue will be flash frozen and stored at −80° C. Harvested tissues are heart, lung, spleen, rest of liver, right kidney, and left kidney. Humanized liver mice strain is UPA HUMANIZED LIVER MICE.

2. Study Schedule
2.1. Animal Receipt Date: Sep. 29, 2021
2.2. Acclimatization/Quarantine End Date: 5 days
2.3. Baseline Procedure Date: No baseline procedures
2.4. Procedure Start Day 0 Date: Tentative: October

Waiting on test material.
2.5. Necropsy Start: On Day 14 post treatment.
2.6. In-Life Study Completion: 14 days post treatment
2.7. Preliminary Report: None required by Sponsor, Data only
2.8. Final Report Issued: None required

3. Test System Information
3.1. Animal Test
   3.1.1. Common Name: Mouse
   3.1.2. Breed/Class: Rodent—uPA humanized liver Mouse
   3.1.3. Number of Animals (by gender): 36 Male, all naïve
   3.1.4. Age Range: 14-19 weeks
   3.1.5. Weight Range: Approx. 20 grams
3.2. Acclimation Period:
   3.2.1. Duration:

All animals will be acclimated for a minimum period of five (5) days prior to release by the Attending veterinarian, at which time the overall health of the animals will be evaluated. Animals which are not released from acclimation will be treated accordingly and further evaluation will be performed prior to release. All records from the acclimation period will remain in the study file.

4. Study Design
4.1. Design Details

This study will have one type of mice, 35 uPA humanized liver mice. Animals will be grouped by treatment type and dosage. Each animal will be treated by subcutaneous injection of test material. (Note: that the injection must be given subcutaneously. The test articles will not be functional if the subcutaneous site is missed, and injection is given within the muscular region or test articles are injected into the vein/bloodstream). Group 1 will have five animals receive a control dose of PBS. Groups 2-7 will receive one dose (10 or 30 mg/kg) with five animals for each dose amount. See study table 1 for details. All animals will be survived for 14 days. At pre-euthanasia, plasma and serum will be collected from all animals, flash frozen and stored at −80° C. At necropsy, two 2 mm biopsy punches will be taken from each site: left, middle and right liver lobes (total 6 biopsies). Three biopsy samples, one from each liver lobe, will be soaked in RNAlater for 15 minutes, flash frozen and stored at −80° C. The remaining three liver biopsies will be placed into one vial, flash frozen and stored at −80° C. All other tissue samples, heart, lung, spleen, rest of liver, right kidney, and left kidney will be flash frozen and stored at −80° C.

TABLE 8
Study Table

| | Number of | | | | Pre- |
|---|---|---|---|---|---|
| | UPA | Treatment | | | Euthanasia |
| | HUMANIZED | Subcutaneous | | Blood | And |
| | LIVER MICE | Injection | Dose | Draw(Day | Necropsy(Day |
| Group | Animals | (Day 0) | mg/kg | 14) | 14) |
| 1 | 5 | PBS | — | Plasma | Pre- |
| 2 | 5 | P29-A28 | 10 | and serum | Euthanasia: |
| | | (15 Config) | | will be | Plasma and |
| 3 | 5 | P29-A28 | 30 | collected | serum |
| | | (15 Config) | | at pre- | collection. |
| 4 | 5 | P29-A277 | 10 | euthanasia | Necropsy: |
| | | (15 Config) | | (Day 14). | 2 mm biopsy |
| 5 | 5 | P29-A277 | 30 | Send | of left, middle |
| | | (15 Config) | | plasma | and right liver |
| | | | | and serum | lobes in |
| | | | | to | separate vials, |
| | | | | Sponsor. | in RNAlater |
| | | | | | for 15 min, |
| | | | | | flash freeze |
| | | | | | and store at |
| | | | | | −80° C. |
| | | | | | 2 mm biopsy |
| | | | | | of left, middle |
| | | | | | and right liver |
| | | | | | all in one vial, |
| | | | | | flash freeze |
| | | | | | and store at |
| | | | | | −80° C. |
| | | | | | Samples of |
| | | | | | heart, lung, |
| | | | | | spleen, rest of |
| | | | | | liver, right |
| | | | | | kidney, and |
| | | | | | left kidney, |
| | | | | | flash freeze |
| | | | | | and store at |
| | | | | | −80° C. |

4.2. Route of Administration

Subcutaneous injection in the scruff. An injection volume of 200 uL.

(Note: that the injection must be given subcutaneously. The test articles will not be functional if the subcutaneous site is missed, and injection is given within the muscular region or test articles are injected into the vein/bloodstream).
4.3. Baseline data collected prior to initiating study.

none

5. Test Article and Ancillary Material Information
5.1. Test Drug 1:
   5.1.1. Identification: P29-A28 (15 Configuration) (see Table 7b)
   5.1.2. Manufacturer: Sirnaomics
   5.1.3. Description: GalNAc-siRNA targeting APOC3 and PCSK9
   5.1.4. Lot/Batch Number: Will be recorded on study materials form.
   5.1.5. Expiration Date: Will be recorded on study materials form.
   5.1.6. Storage Temperature: Ambient
   5.1.7. Bio-Hazard Status: None
   5.1.8. MSDS*: TBD
   5.1.9. Appearance: Clear Liquid
   5.1.10. Dose Information: See Table 8
   5.1.11. Residual Test Article Storage: None
5.2. Test Drug 2:
   5.2.1. Identification: P29-A277 (15 Configuration) (see Table 7b)
   5.2.2. Manufacturer: Sirnaomics
   5.2.3. Description: GalNAc-siRNA targeting APOC3 and PCSK9
   5.2.4. Lot/Batch Number: Will be recorded on study materials form.
   5.2.5. Expiration Date: Will be recorded on study materials form.
   5.2.6. Storage Temperature: Ambient
   5.2.7. Bio-Hazard Status: None
   5.2.8. MSDS*: TBD
   5.2.9. Appearance: Clear Liquid
   5.2.10. Dose Information: See Table 8
   5.2.11. Residual Test Article Storage: None

6. Necropsy and Explant Procedure:

Note: Tissue samples will be taken using separate tools for each individual collection. Tissue harvesting tools will be changed for each tissue sample to prevent cross contamination.

A 2 mm biopsy punch will be taken from the left, middle and right liver lobes. Place biopsy samples into separate 2 ml Eppendorf tubes, with 1.5 ml RNAlater and let soak for 15 minutes, flash freeze then store at −80° C. Three more 2 mm biopsy samples will be taken of the left, middle and right liver lobes all placed together into one 2 ml Eppendorf tubes, flash freeze then store at −80° C. Collected tissue samples: heart, lung, spleen, rest of liver, right kidney, and left kidney will be flash frozen in liquid nitrogen.

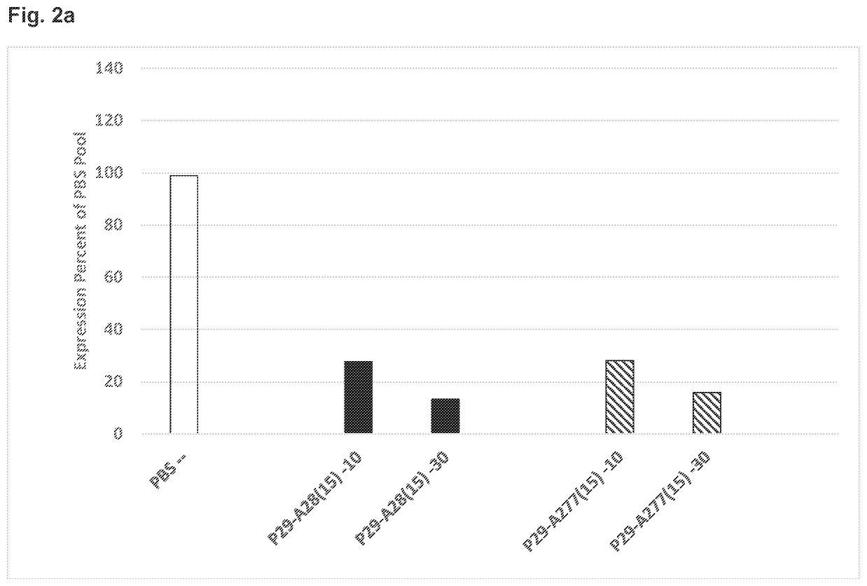

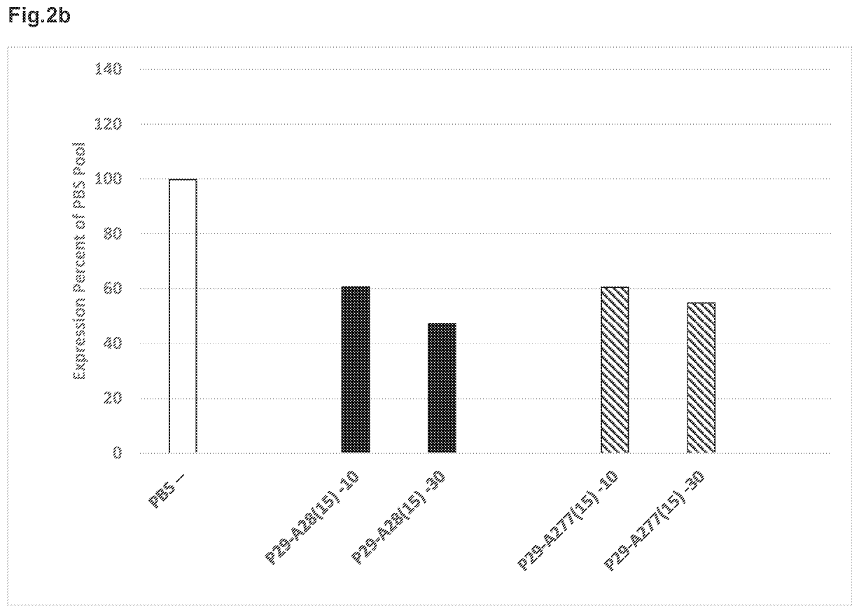

The study results are shown in FIGS. 2a and 2b.

1. A nucleic acid construct comprising at least:
(a) a first nucleic acid portion that is at least partially complementary to at least a first portion of an RNA which is transcribed from a PCSK9 gene;
(b) a second nucleic acid portion that is at least partially complementary to at least a second portion of an RNA which is transcribed from a APOC3 gene;
(c) a third nucleic acid portion that is at least partially complementary to said first nucleic acid portion of (a), so as to form a first nucleic acid duplex region therewith;
(d) a fourth nucleic acid portion that is at least partially complementary to said second nucleic acid portion of (b), so as to form a second nucleic acid duplex region therewith; and wherein the construct
further comprises at least one labile functionality such that subsequent to in vivo administration said construct is cleaved so as to yield at least first and second discrete nucleic acid targeting molecules; and
wherein said labile functionality comprises one or more unmodified nucleotides.

2. The construct according to claim 1, wherein
(a) said first nucleic acid portion has a nucleobase sequence selected from the group consisting of SEQ ID NOs: 1 to 7 and SEQ ID NO: 31;
(b) said second nucleic acid portion has a nucleobase sequence selected from the group consisting of SEQ ID NOs: 8 to 14 and SEQ ID NO: 29;
(c) said third nucleic acid portion has a nucleobase sequence selected from the group consisting of SEQ ID NOs: 15 to 21 and SEQ ID NO: 32; and/or
(d) said fourth nucleic acid portion has a nucleobase sequence selected from the group consisting of SEQ ID NOs: 22 to 28 and SEQ ID NO: 30.

3. The construct according to claim 1, wherein said first nucleic acid portion of (a) is linked to said fourth nucleic acid portion of (d) as a primary structure.

4. The construct according to claim 2, wherein said first and said fourth nucleic acid portions have the nucleobase sequences selected from the group consisting of SEQ ID NOs: 2 and 22; 4 and 24; 5 and 22; 6 and 24; 4 and 22; 6 and 22; 31 and 24; 31 and 22; 4 and 25; 5 and 26; 3 and 28; and 1 and 30.

5. The construct according to claim 2, wherein said second nucleic acid portion of (b) is linked to said third nucleic acid portion of (c) as a primary structure.

6. The construct according to claim 2, wherein said second and third nucleic acid portions have the nucleobase sequences selected from the group consisting of SEQ ID NOs: 8 and 16; 10 and 18; 8 and 19; 10 and 20; 8 and 18; 8 and 20; 10 and 32; 8 and 32; 11 and 18; 12 and 19; 14 and 17; and 29 and 15.

7. The construct according to claim 1, further comprising 1 to 8 additional nucleic acid portions that are respectively at least partially complementary to an additional 1 to 8 portions of RNA transcribed from one or more target genes, and wherein each of the 1 to 8 additional nucleic acid portions respectively form additional duplex regions with respective passenger nucleic acid portions.

8. The construct according to claim 7, wherein said second nucleic acid portion of (b), and said 1 to 8 additional nucleic acid portions, are linked to selected passenger nucleic acid portions as respective primary structures.

9. The construct according to claim 3, wherein said linking represents either (i) an internucleotide bond, (ii) an internucleotide nick, or (iii) a nucleic acid linker portion of 1, 2, 3, 4, 5, 6, 7, 8, 9, or 10 nucleotides, said nucleic acid linker optionally being single stranded.

10. The construct according to claim 9, wherein said internucleotide bond involves at least one of said one or more unmodified nucleotides, wherein optionally cleavage occurs at the 3′ position of (at least one of) said unmodified nucleotide(s).

11. The construct according to claim 1, wherein said first nucleic acid portion of (a), and/or said second nucleic acid portion of (b), and/or said third nucleic acid portion of (c), and/or said fourth nucleic acid portion of (d), are respectively 7 to 25 nucleotides in length, wherein said first nucleic acid portion of (a) and/or said second nucleic acid portion of (b) have a length of 18 to 21, more optionally 18 or 19, and yet more optionally 19 nucleotides, and wherein said third nucleic acid portion of (c), and/or said fourth nucleic acid portion of (d) have a length of 11 to 20, more optionally 13 to 16, and yet more optionally 14 or 15, most optionally 15 nucleotides.

12. The construct according to claim 11, wherein said unmodified nucleotide(s) is/are at any of position 18 to 25, more optionally at any of positions 18 to 21, and most optionally at position 19 and/or the 3′ terminal position of said first nucleic acid portion of (a) and/or of said third nucleic acid portion of (c).

13. The construct according to claim 12, wherein said nucleic acid linker portion is 1 to 8 nucleotides in length, optionally 2 to 7 or 3 to 6 nucleotides in length, more optionally about 4 or 5 and most optionally 4 nucleotides in length, and wherein the construct
further comprises one or more phosphorothioate or phosphorodithioate internucleotide linkages, wherein the.
one or more phosphorothioate or phosphorodithioate internucleotide linkages are located at one or more of the 5′ and/or 3′ regions of said first nucleic acid portion of (a), and/or said second nucleic acid portion of (b), and/or said third nucleic acid portion of (c), and/or said fourth nucleic acid portion of (d), and/or said 1 to 8 additional nucleic acid portions, and/or said passenger nucleic acid portions.

14. The construct according to claim 13 which comprises phosphorothioate or phosphorodithioate internucleotide linkages between at least two adjacent nucleotides of the nucleic acid linker portion, and optionally,
between each adjacent nucleotide that is present in said nucleic acid linker portion.

15. The construct according to any of claim 14, which comprises a phosphorothioate or phosphorodithioate internucleotide linkage linking:
the first nucleic acid portion of (a) to the nucleic acid linker portion; and/or
the second nucleic acid portion of (b) to the nucleic acid linker portion; and/or
the third nucleic acid portion of (c) to the nucleic acid linker portion; and/or
the fourth nucleic acid portion of (d) to the nucleic acid linker portion; and/or
the 1 to 8 additional nucleic acid portions to the nucleic acid linker portion; and/or
the passenger nucleic acid portions to the nucleic acid linker portion.

16. The construct according to claim 1, wherein at least one nucleotide of at least one of the following is modified:
the first nucleic acid portion of (a); and/or
the second nucleic acid portion of (b); and/or
the third nucleic acid portion of (c); and/or
the fourth nucleic acid portion of (d); and/or
to the extent present, the 1 to 8 additional nucleic acid portions; and/or
to the extent present, the passenger nucleic acid portions; and/or
to the extent present, the nucleic acid linker portion.

17. The construct according to claim 16, wherein the modification and/or modifications are individually a sugar, a phosphate, or a base modifications.

18. The construct according to claim 17, where the modification is selected from nucleotides with 2′ modified sugars; conformationally restricted nucleotides (CRN) sugar such as locked nucleic acid (LNA), (S)-constrained ethyl bicyclic nucleic acid, and constrained ethyl (cEt), tricyclo-DNA; morpholino, unlocked nucleic acid (UNA), glycol nucleic acid (GNA), D-hexitol nucleic acid (HNA), and cyclohexene nucleic acid (CeNA), wherein optionally said 2′ modified sugar is selected from 2′-O-alkyl modified sugar, 2′-O-methyl modified sugar, 2′-O-methoxyethyl modified sugar, 2′-O-allyl modified sugar, 2′-C-allyl modified sugar, 2′-deoxy modified sugar such as 2′-deoxy ribose, 2′-F modified sugar, 2′-arabino-fluoro modified sugar, 2′-O-benzyl modified sugar, 2′-amino modified sugar, and 2′-O-methyl-4-pyridine modified sugar.

19. The construct according to claim 18, wherein at least one modification is a 2′-O-methyl modification in a ribose moiety and/or, wherein at least one modification is a 2′-F modification in a ribose moiety.

20. The construct of claim 1, wherein
(a) said first nucleic acid portion is selected from the group consisting of SEQ ID Nos. 33-39;
(b) said second nucleic acid portion is selected from the group consisting of SEQ ID Nos. 40-46;
(c) said third nucleic acid portion is selected from the group consisting of SEQ ID Nos. SEQ ID Nos. 47-53; and/or
(d) said fourth nucleic acid portion is selected from the group consisting of SEQ ID Nos. SEQ ID Nos. 54-60.

21. The construct of claim 1, wherein said construct comprises two strands, wherein the first strand is selected from the group consisting of SEQ ID Nos. 61-114 and the second strand the group consisting of SEQ ID Nos. 115-168; or
said first and second strands are selected from the group consisting of SEQ ID Nos. 110-111, 164-165, and 169-174; or
said first and second strands are selected from the group consisting of SEQ ID Nos. 175-184.

22. The construct of claim 21, wherein the first strand is selected from the group consisting of SEQ ID Nos. 62, 78, 65, 80, 110 to 114, 85, 93, 105, and wherein the second strand is selected from the group consisting of SEQ ID Nos. 116, 132, 119, 134, 164 to 168, 139, 147, and 159.

23. A pharmaceutical composition comprising a construct according to claim 1 and further comprising a physiologically acceptable excipient, diluent, antioxidant, and/or preservative.

24. The pharmaceutical composition of claim 23, wherein said construct is the only pharmaceutically active agent.

25. The pharmaceutical composition of claim 23, wherein said pharmaceutical composition furthermore comprises one or more further pharmaceutically active agents.

26. The pharmaceutical composition of claim 25, wherein said further pharmaceutically active agent(s) comprise an RNAi agent which is directed to a target different from PCSK9 and from APOC3; and/or wherein said agent(s) comprise a lipid-lowering agent distinct from said construct, wherein said lipid-lowering agent is optionally ezetimib; Vascepa; Vupanorsen; statins such as Rosuvastatin and Simvastatin; and/or fibrates such fenofibrate.

27. A method of treating a subject to ameliorate or prevent a disease or disorder comprising: administering to the subject a therapeutically effective amount of the pharmaceutical composition of claim 23, wherein the disease or disorder is a PCSK9 and/or an APOC3-associated disease or disorder, or a disease or disorder requiring a reduction of PCSK9 and/or APOC3 gene expression levels.

28. The method of claim 27, wherein the composition is administered to a subject who is statin-intolerant, or for whom statins are contraindicated.

29. The method of claim 28, wherein said disease or disorder treated comprises:
(a) a PCSK9-associated disease or disorder, or a disease or disorder requiring reduction of low-density lipoprotein (LDL) cholesterol, said disease or disorder optionally being selected from dyslipidemia including mixed dyslipidemia, hypercholesterolemia, heterozygous familial hypercholesterolemia, non-familial hypercholesterolemia; atherosclerosis; and atherosclerotic cardiovascular disease (ASCVD) including myocardial infarction, stroke and peripheral arterial disease;
and/or
(b) an APOC3-associated disease or disorder, or a disease or disorder requiring reduction of APOC3 expression levels, said disease or disorder optionally being selected from dyslipidemia including mixed dyslipidemia; hyperchylomicronemia including familial hyperchylomicronemia; hypertriglyceridemia, optionally severe hypertriglyceridemia and/or hypertriglyceridemia with blood triglyceride levels above 500 mg/dl; inflammation including low-grade inflammation; atherosclerosis; atherosclerotic cardiovascular diseases (ASCVD) including major adverse cardiovascular events (MACE) such as myocardial infarction, stroke and peripheral arterial disease; and pancreatitis including acute pancreatitis.

30. The method of claim 29, wherein the subject is a human.
